(19) United States
(12) Patent Application Publication
(10) Pub. No.: US 2024/0038342 A1
(43) Pub. Date: Feb. 1, 2024

(54) STANDARDIZED PATIENT-PROVIDED DATA AND INTERVENTIONS BASED ON SIGNIFICANCE CODES

(71) Applicant: Mymee Inc., New York, NY (US)

(72) Inventors: Mette Dyhrberg, Brooklyn, NY (US); Christopher V. Beckman, Miami, FL (US); Gillian Sandler, New York, NY (US)

(21) Appl. No.: 18/228,662

(22) Filed: Jul. 31, 2023

Related U.S. Application Data
(63) Continuation-in-part of application No. 17/344,884, filed on Jun. 10, 2021.
(60) Provisional application No. 63/037,551, filed on Jun. 10, 2020.

(51) Int. Cl. G16H 10/60; G16H 10/20; G16H 50/20; G16H 50/70; G10L 15/18; G10L 15/16; G10L 25/66; G10L 15/22; G06F 3/16

(52) U.S. Cl. CPC G16H 10/60; G16H 10/20; G16H 50/20; G16H 50/70; G10L 15/1815; G10L 15/16; G10L 25/66; G10L 15/22; G06F 3/167

(57) ABSTRACT

New systems, devices, methods and other techniques for eliciting, standardizing and recording patient-reported data in clinical healthcare settings are provided. A new form of specialized computer hardware and software control system is provided, applying new machine learning techniques for translating patients' freeform verbalizations and other patient-reported data to standardized significance codes. In some embodiments, the system also translates such significance codes into standardized medical terminology that is more easily actionable by healthcare providers, as part of a new form of personal health record generated by patients (“PHRP”). In some embodiments, health-related data is normalized based on techniques known as translation vectors. Such translation vectors generate standardized significance codes based on common usage of terms by user(s) of the control system (e.g., a cohort of demographically-related patient users), and subsequent entries of such terms by such a user then causes the control system to enter data at least partially defined by the same significance codes.

In some embodiments, patients' personal health records, including language usage and experiences over time, also adjust the translation of such freeform verbalizations into significance codes, and medically-actionable terminology. For example, in some embodiments, such records impact the designation of probable diagnoses, triggers, comorbidities and suggested treatments, as then recorded and suggested to healthcare providers. In some such embodiments, new graphical user interfaces are provided for generating, storing and managing such a PHRP, and generating interventions based thereon.

In some embodiments, the system generates a colloquy, e.g., a script, to aid a healthcare provider in efficiently eliciting significant patient-reported data in a standard format. In some embodiments, such a script is modified and generated in real time, based on the course of the colloquy.

In some embodiments, techniques involving external, objectively sensed healthcare measurements are implemented, which the system may use to validate and calibrate translation vectors between subjective expressions from patients to universal significance codes, based on related expressions from other, similar patients (e.g., from a similar demographic cohort of patients).

### CROSS-REFERENCE TO RELATED APPLICATIONS

The present application is a continuation-in-part of U.S. patent application Ser. No. 17/344,884, titled “Standardized Data Input from Language Using Universal Significance Codes,” filed Jun. 10, 2021. This application claims the benefit of U.S. Provisional Application No. 63/037,551, filed Jun. 10, 2020, titled “Standardized Data Input from Language Using Universal Significance Codes,” which is herein incorporated by reference in its entirety. The entire contents of each of the above applications are hereby incorporated by reference in their entirety into the present application.

### TECHNICAL FIELD

The present invention relates to the field of systems, devices and methods for gathering and managing health-related data and delivering Digital Therapeutics, and, in particular, for standardizing such data with the aid of user interfaces and machine learning techniques.

### BACKGROUND

A persistent problem in medical practice and studies has been how to determine and record subjective patient-reported data that is clinically meaningful. Patients' cognitive abilities, subjective perceptions, and communication skills vary widely, especially when confronted with conditions impairing those functions and a high-stress setting, such as an emergency healthcare setting.

Rigorously tested and standardized approaches to generating actionable patient-reported data have been developed, which attempt to control that variability. For example, in 2004, the Patient-Reported Outcomes Measurement Information System (“PROMIS”) was initiated at the National Institutes of Health (“NIH”) to develop more standardized recordings of key health indicators and symptoms from patients. Generally speaking, PROMIS involves rigorous standardized questioning methods, based on patient response theory, to generate more meaningful verbal data from the patient, in the form of measurements known as “PROMIS scores.” A wide range of health-related data can be generated by such methods, such as PROMIS scores for pain, fatigue, inflammation, range of function, and general wellbeing. In clinical studies, PROMIS scores can be used to measure data regarding main hypotheses of a study (“primary endpoints”) and other data relevant to such studies (“secondary endpoints”). PROMIS score methodology typically involves brief self-reported measures from patents from a narrow selection of graded options (e.g., rating pain on a scale of 1 to 10, 1 being moderate discomfort, and 10 being the most excruciating pain ever experienced by the patient).

Recently, Computer Adaptive Testing (CAT) methods have been developed, which present targeted questions, homing in on a patient's likely outcome levels, and presenting fewer options for the patient to consider and choose from. CAT has been proven to increase the speed and brevity of testing for PROMIS scores.

PROMIS Scores often provide clinically relevant directional information, and aid in treatment. In addition to PROMIS, several similar interpretive questionnaire initiatives and programs for patient reported data have been promulgated worldwide. Useful as PROMIS scores and similar techniques for obtaining patient-reported data, their self-reported, subjective nature, and variability in patients (discussed above) remains a challenge.

For example, questions seeking a simple 1-10 scale for pain do not gather any meaningful characteristics of that pain other than “level.” This can confuse and frustrate patients in many instances, when the location, persistence, sharpness, inflammation, or other characteristics, and changes therein, seem far more significant than level at that moment.

Compounding these problems, healthcare providers face increasingly serious time and productivity pressures in modern medicine, and often neglect to follow PROMIS methodology, or to record other important notes provided by patients. Even when following rigorous (e.g., PROMIS score) questioning techniques, healthcare providers often fail to elicit or record clinically meaningful data.

Some patients manage to express clinically-relevant information in a freeform manner, despite those pressures. However, such expressions are sometimes not recorded, recorded incompletely, changed and substantively altered by the healthcare provider, with or without follow-up questioning. Differences in patient backgrounds and personality, including cultural, language usage, educational, and expressive differences, and those of the particular healthcare provider often lead to additional misunderstandings, resulting in imprecise patient records and poorer patient health assessments.

There remains a long-felt, unmet need for more reliable testing and recording methods for patient-reported data.

Many of the techniques and systems set forth in the present application are implemented, at least in part, with networked, portable computers, such as personal digital assistants (“PDAs”). PDAs allow a user to record and manage personal information, and they have been available in some form for decades. For example, as early as the 1970s, small digital wristwatches allowed users to perform personal computing, such as financial arithmetic, and storing information related to personal contacts, such as names, addresses and phone numbers. The now virtually ubiquitous smartphones can be thought of as modern PDAs, capable of sophisticated, highly secure communications over a network, and running some of the most complex computer programs. Specialized software designed to be run on smartphones, known as “Apps,” allow users to provide and receive a wide variety of data, and perform a wide variety of functions based on those data, ranging from online banking to digital gaming.

Some such Apps relate to personal health and/or fitness management (a.k.a., “Health and Fitness” Apps. For example, at least some such Apps, and some other software, are known as “Digital Health” software, which, as used in the present application, means software: aiding a user(s) (and/or their caregivers, and/or friends and family) in managing the user's(s') health- and/or fitness-related: i. behavior; ii. environment; and/or iii. information. Some such Digital Health software is used with associated hardware, such as a heart rate monitor, blood pressure sensor, or other health-related sensors and actuators. Some Digital Health software and hardware falls within the definition of “Digital Therapeutics.”

As used in the present application, “Digital Therapeutics” means: evidence-based therapeutic interventions, driven by software, to: a) prevent, manage and/or treat an adverse and/or unwanted physical, mental and/or behavioral illness, disorder and/or condition; and/or b) create a beneficial and/or desired physical, mental and/or behavioral illness, disorder and/or condition.

Some Health and Fitness Apps, and some Digital Health Apps, may be “Telehealth” software, meaning that the App enables a doctor or other caregiver to provide a remote examination of and/or consultation to a user (e.g., a patient). Similarly, some Health and Fitness Apps, and some Digital Health Apps, may be software related to “Adherence,” meaning that the software enables a user and/or their caregiver(s) to monitor and aid the user in maintaining a regimen of pharmaceutical(s) or other nutrient(s), environmental factor(s) or behavioral intervention(s) in accordance with a plan.

It should be noted that some of the disclosures set forth as background, such as, but not limited to, the above language under the heading “Background,” may not relate exclusively to prior art and the state of the art in the field(s) of the invention, and should not be construed as an admission with respect thereto.

### SUMMARY

New systems, devices, methods and other techniques for eliciting, standardizing and recording patient-reported data in clinical healthcare settings are provided. A new form of specialized computer hardware and software control system is provided, applying new machine learning techniques for managing patient-reported data, and developing standardized data related to the patient-reported data, as part of a new form of personal health record (“PHR”) generated, at least in part, by a patient (a.k.a., a “PHRP”). In some such embodiments, new graphical user interfaces (“GUIs”) are provided for generating, storing and managing such a PHRP, and generating interventions based on such a PHRP.

In some embodiments, health-related data, such as, but not limited to, such patient-reported data, are normalized based on significance maps and translation vectors. In some embodiments, standardized significance codes are defined and implemented, based on the usage of terms by user(s) of the control system, and subsequent entries of such terms by a user then causes the control system to enter data at least partially defined by the significance codes. In some embodiments, such translation vectors generate standardized significance codes based on common usage of terms by user(s) of the control system (e.g., a cohort of demographically-related patient users), and subsequent entries of such terms by such a user then causes the control system to enter data at least partially defined by the same significance codes. And, in some embodiments, such translation vectors generate standardized significance codes, at least in part, based on a patient user's related medical history over time, as recorded through the system, as well as related medical histories between such a patient user and one or more other patient users of the system, using the same or similar terms.

In some embodiments, verbal expression recordation and analysis systems and methods are provided. In some such embodiments, a system including specialized hardware and software, and a freeform verbal expression recordation and analysis software module, is provided, which allows a user to express concerns in their own words, generating a freeform expression key term tag cloud, with patient-centered priority ranking scores for each keyword. In some embodiments, the freeform verbal expression software module generates a map of potential symptoms and symptom characteristics, each with a probability ranking relative to one another, for the patient, based on her recorded freeform verbal expression. In some embodiments, the system generates standardized medically-relevant concepts and terms related to patient care, such as a list of potential diagnoses and interventions, based, at least in part, on such a map and/or probability ranking. In some embodiments, with respect to such potential diagnoses, such a probability ranking is an expression of relative probability of each potential diagnosis, each in comparison to the other, as to their likelihood of relevance to the patient. And, in some embodiments, with respect to such potential interventions, such a probability ranking is an expression of relative probability of success of potential intervention, each in comparison to the other, when executed on behalf of the patient.

In some embodiments, the system generates a decision tree related to the seriousness of misdiagnosis and failure to treat each potential diagnosis and/or to perform each intervention. In some such embodiments, the system ranks each of the potential diagnoses and interventions for physician attention, in accordance with the second probability ranking and the decision tree related to the seriousness of misdiagnosis and failure to treat each diagnosis, if actually present. However, in some embodiments, the second probability ranking and the decision tree are adjusted in an algorithm (e.g. a machine learning-created algorithm) by at least one coefficient related to the likelihood of such a diagnosis (e.g., for the patient, or a demographic cohort including the patient). In various embodiments, each such decision tree, potential diagnosis, and diagnosis probability ranking, may be generated with the aid of an algorithm and, in some such embodiments, new machine learning techniques. Similarly, in related embodiments, the system generates a list of most probable diagnoses, comorbidities symptom and condition triggers (e.g., triggers of inflammation) based on similar decision trees and algorithms.

In some embodiments, the system generates a colloquy, e.g., a script, to aid a healthcare provider in efficiently eliciting significant patient-reported data in a standard format. In some embodiments, such a script is modified and generated in real time, based on the course of the colloquy.

In some embodiments, techniques involving external, objectively sensed healthcare measurements are implemented, which the system may use to validate and calibrate translation vectors between subjective expressions from patients to universal significance codes, based on related expressions from other, similar patients (e.g., from a similar demographic cohort of patients).

As mentioned above, the techniques may include methods and systems, in some embodiments. In some embodiments, such systems include computer hardware and software, including non-transitory machine-readable media with executable instructions. When executed by computer hardware, the instructions may cause the systems to carry out any or all of the methods set forth in this application.

These and other aspects of the invention will be made clearer below, in other parts of this application. This Summary, the Abstract, and other parts of the application, are for ease of understanding only, and no part of this application should be read to limit the scope of any other part, nor to limit the scope of the invention, whether or not it references matter in any other part.

Further aspects of the invention will be set forth in greater detail, below, with reference to the particular figures.

### BRIEF DESCRIPTION OF THE DRAWINGS

The features and advantages of the example embodiments of the invention presented herein will become more apparent from the detailed description set forth below when taken in conjunction with the following drawings.

FIG. 1 is a perspective view of an example clinical healthcare environment, including an example healthcare provider eliciting patient reported data and standardizing and recording personal health records with the aid of a personal health record generation system, in accordance with some embodiments.

FIG. 2 is a front view of an example local control system, display and graphical user interface of a personal health record generation system, in accordance with some embodiments.

FIG. 3 is a process flow diagram, setting forth several example steps that may be undertaken by a control system (such as the example control system set forth below, in reference to FIG. 5) implementing some aspects of the present invention, according to some embodiments.

FIG. 4 is a front view of an example GUI, implementing some example aspects of the present invention related to monitoring and gathering data related to a user (e.g., patient-reported data/behavior), in accordance with some embodiments.

FIG. 5 is a schematic block diagram of some example elements of an example control system that may be used to implement various aspects of the present invention, some of which aspects are described in reference to FIGS. 1-4 and 6-9 of this application, in accordance with some embodiments.

FIG. 6 is a perspective view of an example environment in the process of being monitored by an example imaging sensor, which may be controlled by a control system including computer hardware and software (such as any of the control systems set forth in this application), in accordance with some embodiments.

FIG. 7 is a perspective view of an example athletic environment, including a view of the same example patient, discussed above, being monitored by an example imagining sensor (which may be an imaging sensor similar in nature to that set forth above, in reference to FIG. 6), of personal health record generation system, in accordance with some embodiments.

FIG. 8 is a front view of the same example local control system, display and graphical user interface of a personal health record generation system, but displaying additional user interface aspects, based on additional patient-reported data recorded by the system at a later time, based on additional experience with the patient, in accordance with some embodiments.

It should be noted that the figures referenced above are examples only of the wide variety of different embodiments falling within the scope of the invention, as will be readily apparent to those skilled in the art. Thus, any particular size(s), shape(s), proportion(s), scale(s), material(s) or number(s) of elements pictured are illustrative and demonstrative, and do not limit the scope of invention, as will be so readily apparent.

### DETAILED DESCRIPTION

The example embodiments of the invention presented herein are directed to systems, devices and methods for eliciting, standardizing and recording health-related data with new, specialized information technology, which systems, devices and methods are now described herein. This description is not intended to limit the application of the example embodiments presented herein. In fact, after reading the following description, it will be apparent to one skilled in the relevant art(s) how to implement the following example embodiments in alternative embodiments.

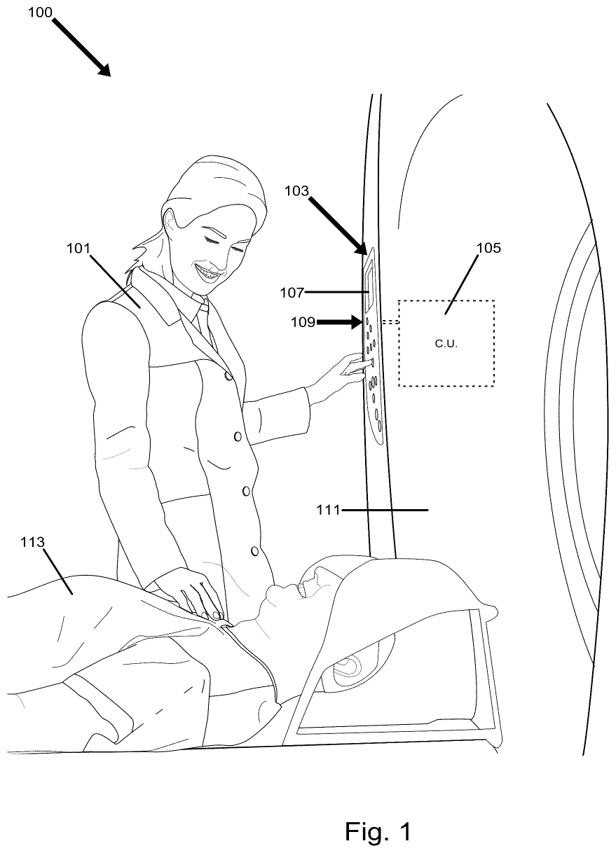

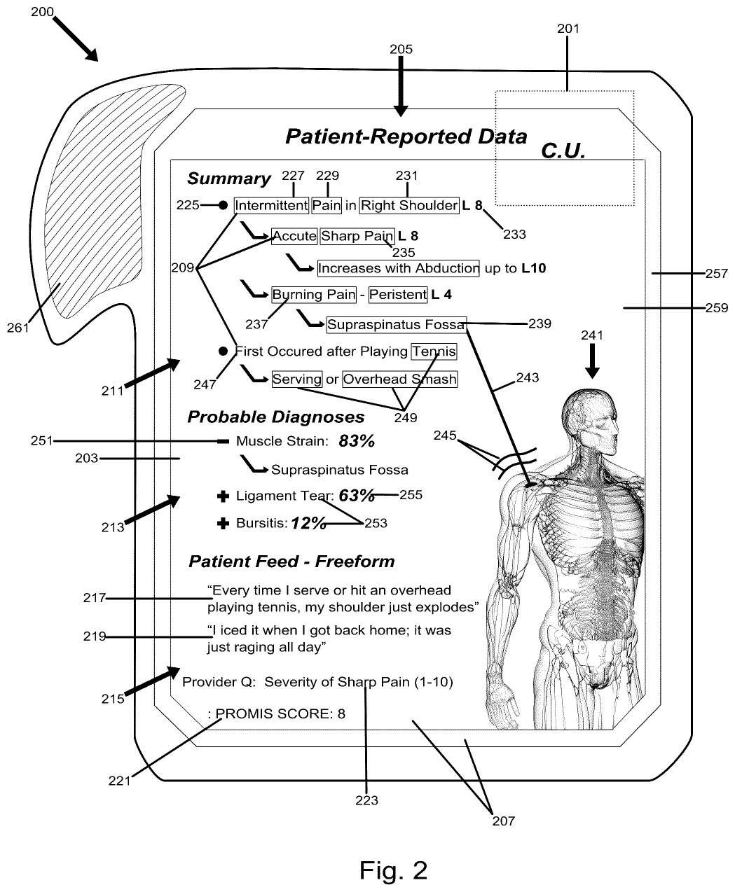

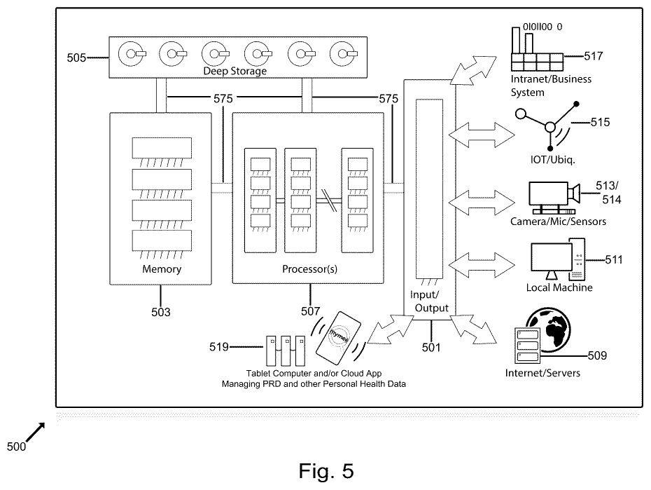

FIG. 1 is a perspective view of an example clinical healthcare environment 100, including an example healthcare provider 101 eliciting patient-reported data (“PRD”) and standardizing and recording personal health records with the aid of a personal health record generation system, in accordance with some embodiments. The example healthcare provider 101 is shown using a wall-mounted personal computing device (a “PCD”), shown as wall-mounted PCD 103, which includes computer software and hardware, e.g., within a local computer unit 105. As with other computing devices set forth in the present application, in some embodiments, PCD 103 comprises, or is comprised within, a control system including computer hardware and software, which may be the same as, or similar to, the example control system set forth below as control system 500, in FIG. 5. Also, at the outset, it is important to note that, although the example of a wall-mounted PCD 103 is provided, any of the techniques set forth herein may be practiced, instead or in addition, with other forms of computing devices and PCDs and other devices comprising, or comprised within, such a control system. For example, in some embodiments, a user may be holding or otherwise interacting with another form of personal electronics computing device comprising and/or comprised within such a control system, such as a personal digital assistant device (“PDA”), desktop computer and/or external peripheral device which, in some embodiments, are not handheld (e.g., a computing device comprising or comprised within ear-mounted audio devices (such as wireless earphone(s) with microphone(s)), smartwatch(s), a wall-, ceiling- or otherwise environmentally-mounted display device(s), and/or any number of ambient intelligence, augmented reality, mixed reality and/or display device(s)), any of which computing devices may carry out each of the techniques set forth herein with respect to wall-mounted PCD 103, in various embodiments. Another example of such a device, namely, a portable tablet computer, is provided below, in reference to FIG. 2. In any event, as also discussed in greater detail below, in some embodiments, such a control system is comprised within a larger system for eliciting, standardizing and recording patient-reported health-related data with new, specialized information technology, embodiments of which will be discussed in greater detail herein.

Regardless of the form of computing device used, in some embodiments, such a system, including such a control system and computing device, may create a user interface (such the example shown as graphical user interface (“GUI”) shown in reference to FIG. 2), for aiding a professional healthcare provider (such as example healthcare provider 101), in eliciting, standardizing and recording patient-reported data and personal health records, and managing medical interventions based thereon, as well as managing other health-related information, in some embodiments. As just some examples, some such health information includes, but is not limited to: biometrics; vital signs; genomic information; proteomic information; genotype information; phenotype information; biomarkers; exercise-related information; activity-related information; environmental information; dietary information; drug and/or drug treatment regimen adherence information, other adherence-related information; behavioral information; and subjective symptomatic information. In some embodiments, such as that pictured, such a user interface is included and presented on a graphical display, such as example PCD display 107, which, as shown, may be an integrated sub-component of wall-mounted PCD 103, in some embodiments. More specifically, as pictured, PCD display 107 is included in a GUI area 109 of PCD 103 which, in turn, may be attached to, part of and/or integrated with, a medical diagnostic imaging or other scanning device 111, in some embodiments. In some embodiments, PCD display 107 may present several patient-reported data indicators (not pictured in the present figure), such as example PRD indicators 209, discussed below, in reference to FIG. 2. Generally speaking, such PRD indicators 209 indicate patient-reported data and other healthcare-related data currently elicited and recorded with the aid of the system for a particular patient, such as the example patient 113 pictured. In addition, in some embodiments, PRD indicators 209, or other GUI tools and/or indicators also guide such a healthcare provider and/or patient, aiding in eliciting such patient-reported data from a patient. In some embodiments, example patient 113 may be assigned to a HIPPA-compliant data account (a “patient account”) through or in connection with the system. In some such embodiments, a healthcare provider, such as example healthcare provider 101, who, in various embodiments, may be a duly licensed doctor, private nurse practitioner, registered nurse, health coach, nutritionist, nurse's aide, health technician, orderly, medical food service employee, hospital administrative staff, robotic or other autonomous artificial intelligence sub-system or module, or another form of healthcare provider, may have administrative privileges to access such a patient account, and thus have access to view and use each of PRD indicators 209, among other GUI tools provided through the system.

Once logged in as an administrator of such a patient account, in some embodiments, healthcare provider 101 may begin eliciting patient-reported data from the patient, with the aid of the system, for recordation and carrying out additional methodological steps. In some embodiments, the system may directly elicit, and/or assist the healthcare provider in eliciting, patient-reported data from the patient, to be so recorded and used in subsequent methodological steps.

For example, in some embodiments, the healthcare provider may initiate a specific colloquy with the patient, to elicit verbal responses on particular topics related to the patient's health and treatment.

As also discussed elsewhere in this application, such a colloquy and such elicited verbal responses may be conducted rigorously, according to a question format, question substance and/or pattern of questions (hereinafter, a “script”) generated by the system based on initial intake information provided by the patient and/or the patient's previously recorded personal health record(s). For example, in some embodiments, the system may generate a unique script for the patient, having a unique question format, question substance and/or pattern of questions based on symptom(s) and condition(s) recorded in the patient's initial intake data and/or previously-recorded personal health records. In some embodiments, the question format, substance and the pattern of questions each may be unique to that script, patient and/or time in which it is generated and/or administered to the patient (e.g., in some embodiments, changing with user/system experience, in accordance with patient-reported data gathered over time from the patient). However, in some other embodiments, only two of the above-mentioned: a) question format, b) question substance or c) pattern of questions is unique to that script, patient and/or time in which it is generated and/or administered to the patient. And, in some other embodiments, only one of the above-mentioned: a) question format, b) question substance or c) pattern of questions is unique to that script, patient and/or time in which it is generated and/or administered to the patient. In some embodiments, such a script may be dynamically generated, with the above-mentioned question format, question substance and pattern of questions changing as the colloquy progresses and develops between the healthcare provider and patient. In some such embodiments, such a script may change in real time. For example, in some embodiments, such a script is at least partially dynamically generated or otherwise created (e.g., by combining some of a plurality of pre-written questions from a library of questions stored in memory of the system), based on new patient-reported data, as it is being provided by the patient in such a system-managed colloquy. As another example, in some embodiments, such a script is dynamically generated based on ad hoc alterations of the script by the healthcare provider. For example, in some such embodiments, the healthcare provider may alter questions and/or the question order of the script, e.g., based on additional diagnostic or other medical ideas that occur to the healthcare provider, and such a script is at least partially generated based on the substance of such alterations and patient-reported data elicited by them. As another example, in some embodiments, the healthcare provider may ask the patient unscripted questions, different from those set forth in the script generated by the system, and such a script is at least partially generated based on the substance of such unscripted questions, and patient-reported data elicited by them.

In some embodiments, and as also discussed elsewhere in this application, the system may maintain a library of significance codes, e.g., in a database, each of which significance codes corresponds with a particular, definite patient sensation, mental state, condition, symptom and/or diagnosis significance. In some such embodiments, such significance codes may be maintained in a standard, structured database format (e.g., csv, SQL, etc.). In some embodiments, such significance codes may be maintained in a semi-structured database (e.g., JSON).

And, in some embodiments, one or more significance code(s) may be selected by the system and/or the healthcare provider, and recorded as relating to the patient (i.e., as a part of PHRP, based on patient-reported data initially elicited from the patient).

In some embodiments, the system may validate such a significance code that has been selected by the system as being indicated by the patient, healthcare provider, and/or other PHR, by determining whether such a significance code has been or is actually being correctly applied to the patient, using objective evidence correlated with a universal significance of that significance code.

For example, in some embodiments, as a healthcare provider, such as healthcare provider 101, engages in a colloquy with the patient, as discussed above, and presents one or more question(s) to the patient related to a patient-reported or other subjectively perceived symptom (for example, patient-reported chronic pain indicated in the patient's previous health records), the patient may undergo objective physiological monitoring managed by the system to evaluate, validate or qualify candidate significance codes to be recorded by the system as related to that patient-reported or other subjectively perceived symptom. For example, in some embodiments, the system includes a significance code qualifying subsystem, including a perception monitoring subsystem and computer hardware and software that objectively monitors indicators of the actual presence of patient-reported data and determines whether candidate significance codes should be, or remain, so recorded by the system as related to that patient-reported or other subjectively perceived symptom. In some embodiments, such a significance code qualifying subsystem, and such a perception monitoring subsystem, tests for the presence of one or more patient-reported or other subjectively perceived symptoms potentially relevant to the patient, based on the patient's PHR(s), and/or selected by a healthcare provider as relevant to, or potentially relevant to, the patient's health.

As some examples, in various embodiments, the significance code qualifying subsystem, with the aid of the perception monitoring subsystem, carries out any or all of the following vital sign or other physiological tests for quantitative analysis, among other possible physiological tests: blood pressure, heart rate, blood oxygen or carbon dioxide saturation, galvanic skin response, local chemical testing or biopsy testing, nerve conduction monitoring, and/or a brain activity scan(s). In some embodiments, some of the tested quantitative levels, as the case may be, may be tested at a particular point in time, or over time (e.g., in “area under the curve” analysis) or for changes therein, over time.

To continue with the example of testing for the presence and amount of perceived pain for the patient, in some embodiments, the significance code qualifying subsystem and perception monitoring subsystem may test for involuntary and/or objective indicators of the presence or absence of the particular pain level reported verbally by the patient (e.g., in such a colloquy as discussed above), which pain level may be initially linked with a candidate significance code first recorded by the system (e.g., pain reported as level 8 on a scoring system of 1-10, 10 being the highest level of pain experienced by the patient previously, is recorded as having a candidate significance code corresponding with such a pain level of 8 out of 10 for a particular patient cohort or other demographic population). In some embodiments, the significance code qualifying subsystem and perception monitoring subsystem then tests if monitored and recorded involuntary and/or objective indicators for the patient are highly correlated (e.g., a correlation of 0.75 or higher, or 0.85 or higher, in some example embodiments) with such involuntary and/or objective indicators monitored and recorded for other patients (e.g., within a demographic cohort of the which the patient is a part) reporting the same level of pain as PRD. For example, in some embodiments, the system tests whether the patient's pattern of galvanic skin response, nerve conduction pattern (e.g., at the dorsal trunk receiving pain signals from the site of pain reported by the patient) and/or a pattern of brain activity while reporting such a pain level matches a pattern of galvanic skin response, nerve conduction pattern and/or a pattern of brain activity highly correlated with the reported pain level in a cohort or other demographic population including the patient, and, if so, a significance code substantiating that level of pain may be determined to be validated by the system, linked in the patient's PHR as a record of the patient's pain level at that point in time, and finally recorded as applicable to the patient, in the patient's PHR (e.g., as the patient's actual, validated significance code for pain level). In some embodiments, in addition to or instead of that final recordation, the system may modify the significance code, and/or the candidate significance code, by recording and linking to the significance code a coefficient incorporating the level of correlation determined by the system. In some embodiments, the system may also record an identifier of the type of testing conducted, as the source of the significance code and/or correlation finally recorded, establishing an audit trail for the creation of the significance code finally recorded or appended with an indicator of the level of correlation. In some embodiments, the system may then modify diagnoses and select or suggest treatments, or other interventions for the healthcare provider, based on such significance codes finally recorded, modifications thereto and/or levels of correlation.

In some embodiments, a healthcare provider, such as example healthcare provider 101, may override a significance code so finally recorded for the patient, instead assigning a significance code on the same symptom or other subject that, in her professional judgment, is more accurate for the patient. In some such embodiments, the system maintains a recorded audit trail of any such overriding or similar change, however, identifying the healthcare provider who made the decision to override the significance code, the change in significance code caused by the overriding, the time, and/or reasoning the change in significance code.

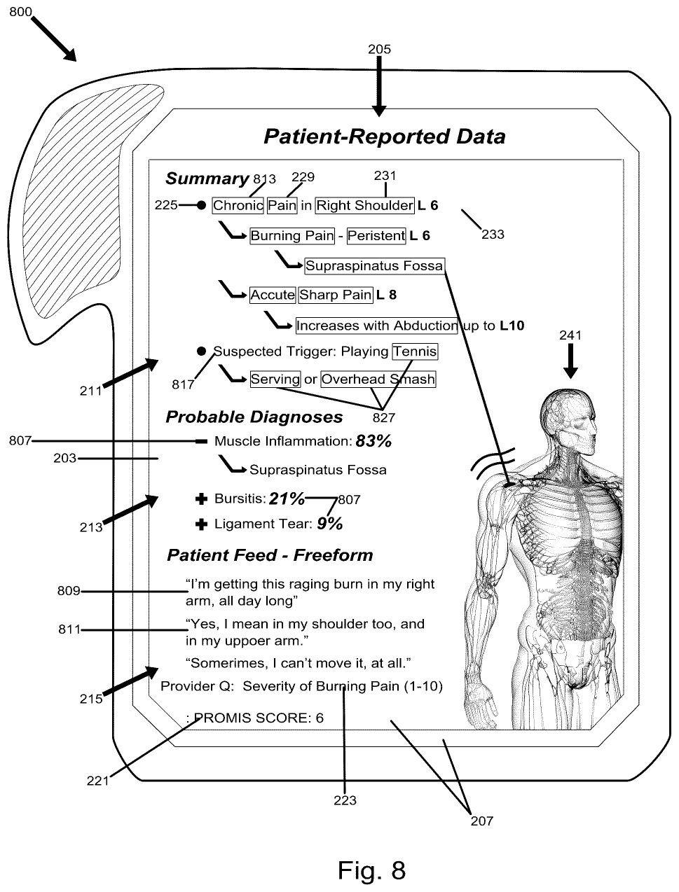

As mentioned above, to assist the healthcare provider, and additional healthcare providers at a later time, in viewing and understanding significance codes as they as so finally recorded, and the nearest equivalent standard medical terminology, specialized GUI's may be implemented by the system. For example, such example GUIs are discussed immediately below, in reference to FIGS. 2 and 8.

FIG. 2 is a front view of an example personal computing device (a “PCD”), namely, example portable tablet computer 200, including a local control system 201, a PCD display 203 and an example graphical user interface of a personal health record generation system, in accordance with some embodiments. As with other control systems of PCDs, and other local control systems set forth in the present application, in various embodiments, local control system 201 may include, or be included within, a system for generating (e.g., eliciting, standardizing and recording) patient-reported data (e.g., personal health records) (the “system”), in accordance with aspects set forth in this application. As set forth elsewhere in this application, such a system may include specialized computer hardware and software of a control system that aids in eliciting, standardizing, and recording patient-reported data, creating personal health records, and managing medical interventions based thereon. Examples of such a control system, including such specialized computer hardware and software, are provided in reference to FIG. 5, below. Although the example of a portable tablet computer 200 is provided, any of the techniques set forth in this application may be practiced, instead or in addition, with other forms of PCDs and other devices comprising, or comprised within such a control system. For example, in some embodiments, a user may be holding or otherwise interacting with another form of personal electronics comprising and/or comprised within such a control system, such as a personal digital assistant device (“PDA”), desktop computer and/or external peripheral devices which, in some embodiments, are not handheld (e.g., a wall-, ceiling- or otherwise environmentally-mounted display device, and/or any number of ambient intelligence, augmented reality, mixed reality and/or display devices), any of which may carry out each of the techniques set forth herein with respect to portable tablet computer 200, in various embodiments.

Among other use environments, as discussed above, a PCD such as example portable tablet computer 200 may be used by a healthcare provider and/or patient to elicit, record and evaluate patient-reported data in a clinical healthcare environment (such as, but not limited to, example clinical healthcare environment 100, discussed above). More specifically, portable tablet computer 200 may create a graphical user interface (“GUI”) such as the example shown as personal health record GUI 205, which, in some embodiments, may be presented on a display, such as the example interactive touchscreens 207 of display 203 and, more generally, of portable tablet computer 200. As mentioned above, in some embodiments, such a GUI may aid a healthcare professional in eliciting, standardizing, and recording patient-reported data, creating personal health records, and managing medical interventions based thereon to aid the patient subject to thereto. In some embodiments, such a GUI also aids such a healthcare provider and patient by presenting, analyzing and managing auxiliary medical, fitness and other health information. As just some examples amid unlimited possibilities, some such health information includes, but is not limited to: biometrics; vital signs; genomic information; proteomic information; genotype information; phenotype information; biomarkers; exercise-related information; activity-related information; environmental information; dietary information; drug and/or drug treatment and adherence information; dietary regimen and adherence information; vitamin and mineral administration information; hydration and intravenous (“IV”) fluids information; other adherence-related information; cognitive and behavioral information; testing information; and subjective symptomatic information. The above listing is illustrative of the virtually unlimited number and types of auxiliary medical, fitness and other health information that may be presented, managed and used by the system, and users thereof through such a GUI in a virtually unlimited number of alternative embodiments that fall within the scope of the present application, and does not limit the scope of invention set forth therein, as will be clear to those of ordinary skill in the art.

In some embodiments, such as that pictured, personal health record GUI 205 may present several patient-reported data indicators, such as example PRD indicators 209. Generally speaking, such PRD indicators may be grouped and presented in several discrete sub-sections of GUI 205, such as example summary GUI subsection 211, probable diagnoses GUI subsection 213 and freeform patient feed GUI subsection 215, in some embodiments (as pictured). Also generally speaking, in some embodiments, such GUI subsections may be arranged, at least partially, as an “inverted funnel” of information, in the sense that GUI subsections toward the bottom of the GUI (and in the perspective of the figure) relate information in a form more closely matching verbal or other patient input. And GUI subsections toward the top of the GUI (and, again, in the perspective of the figure) include a more “cleaned” form of then raw data generated (e.g., verbally) by the patient, and including, and modified by, additional standardized and analytical information, such as significance codes, translation vectors and significance maps, as discussed in greater detail elsewhere in this application.

Thus, beginning at the bottom of the page, the freeform patient feed GUI subsection 215 represents the widest part in the patient-reported information funnel, displaying patient-reported data in its original form, as provided verbally from the patient. For example, in some embodiments, such a freeform patient feed GUI subsection includes sub-tools presenting data representative of original data input from the patient (e.g., provided in response to a questionnaire or oral colloquy with a healthcare provider, as discussed above). For example, in some embodiments, patient quotation sub-tools, such as example first patient quotation sub-tool 217 and example second patient quotation sub-tool 219, are provided. In some such embodiments, such a patient quotation sub-tool may present a verbatim transcript of verbal language so provided by the patient. In some such embodiments, such patient quotation sub-tools provide selected quotes of particular phrases or terms provided by the patient, which the system initially determined to have potential relevance to the health of the patient. And, in some such embodiments, such patient quotation sub-tools provide selected quotes of particular phrases or terms provided by the patient, which the system initially determined to have potential relevance to the health condition of the patient, or suspected health condition (e.g., which motivated the patient's visit to the healthcare professional, in some embodiments.) In some embodiments, as pictured, such patient quotation sub-tools include ordinary verbiage of the patient, related from to the healthcare provider and/or system, and recorded by the system in a verbatim and/or raw, unaltered format. Thus, as pictured, in some embodiments, example first patient quotation sub-tool 217 relates a first discrete verbal phrase or term (i.e., a complaint) by the patient that his “every time I serve or hit an overhead playing tennis, my shoulder just explodes.” And, similarly, as pictured, in some embodiments, example second patient quotation sub-tool 219 relates a second discrete verbal phrase or term by the patient that his “I iced it when I got back home; it was just raging all day.” Although, in some embodiments, such patient quotation sub-tools are provided in a raw, original, unaltered format, in some embodiments, they are limited in number, and selected for relevance to a particular disease, health condition, or health concern to which the patient's visit to the healthcare provider relates. More specifically, in some embodiments, the system may implement a recordation and analysis module, including specialized computer hardware and software, to identify and prioritize different freeform verbal data, depending on its relatedness to standardized health information, such as particular diagnoses, symptoms, health conditions, comorbidities and triggers related to terms and phrases used by a patient. For example, if the patient is visiting an orthopedic surgeon as that healthcare provider and, during an intake process, identified shoulder pain as the reason for the visit, the system may only include such patient quotation sub-tools in GUI 205 (within freeform patient feed GUI subsection 215) that are determined to relate to a health assessment, potential diagnosis and/or treatment of such shoulder pain, in some such embodiments. In this sense, the healthcare provider is able to view the original, underlying material supporting additional GUI tools (e.g., PRD indicators in other subsections of GUI 205, above freeform patient feed GUI subsection 215, as set forth in the present figure), to assess the underpinnings for such additional GUI tools and determine, in her professional judgment, whether the information indicated therein (e.g., potential diagnoses and/or treatment steps, in various embodiments) is valid based on that underlying material, or if, instead, a deviation therefrom may be appropriate. In other words, the GUI sub-tools with in GUI freeform patient feed GUI subsection 215, such as first patient quotation sub-tool 217 and second patient quotation sub-tool 219, provide context for potential diagnoses and treatments, both provided by the system and the healthcare provider, as set forth elsewhere in this application and, more specifically, below.

The example of a patient quotation sub-tool, such as first patient quotation sub-tool 217 and second patient quotation sub-tool 219, is only one of unlimited possible forms of sub-tools presenting data representative of original data input from the patient, any of which may also, or alternatively, be presented within freeform patient feed GUI subsection 215, in various embodiments. For example, in some embodiments, one or more GUI sub-tools may present patient-reported data input from the patient in response to standardized verbal inquiries (e.g., PROMIS scores, as discussed in this application). Thus, for example, in some embodiments, freeform patient feed GUI subsection 215 may present a record of one or more such response(s) (i.e., PROMIS score(s)) so reported by the patient, in an example standardized answer PRD indicator 221. In some embodiments, such a standardized answer PRD indicator presents a patient-provided multiple-choice answer (e.g., as pictured, a PROMIS Score of 4). In some embodiments, as pictured, additional context for such a patient-provided multiple-choice answer may be provided, in some embodiments, such as the substance (as pictured) or exact verbiage, in various embodiments, of the healthcare provider's verbal question or other elicitation of the patient-provided multiple-choice answer, in a contextual component, such as example contextual component 223, of example standardized answer PRD indicator 221. Thus, as pictured, example contextual component 223 shows that the healthcare provider inquired regarding the severity of a “burning” pain reportedly experienced by the patient, on a scale of 1-10, to which the patient provided the answer of 4.

It should be noted that, in some embodiments, as pictured, GUI tools and sub-tools thereof may be provided with visual representations of relationships indicating a substantive relationship between them. For example, as pictured, example standardized answer PRD indicator 221 is presented below, and to the right, of contextual component 223 (presenting the inquiry), to which it relates, and includes a leading symbol (namely, a colon symbol, shown as “:”) indicating that it follows as an answer to that inquiry. Other forms of visual representations of relationships between PRD indicators will be discussed below, in relation to other GUI subsections of GUI 205.

As discussed elsewhere in this application, in some embodiments, the system may implement a recordation and analysis module, including specialized computer hardware and software, to identify and prioritize different freeform verbal data, depending on its relatedness to standardized health information, such as particular diagnoses, symptoms, health conditions, comorbidities and triggers related to terms and phrases used by a patient. And, also as discussed elsewhere in this application, in some embodiments, the system normalizes freeform verbal data provided by a patient by using universal significance codes and translation vectors based on significance maps. For example, and as so discussed elsewhere in this application, in some embodiments, standardized significance codes are defined and implemented, based on the usage of terms by the patient and other users of the control system, over time, and verbal data reported by the patient then causes the control system to enter additional standard data linked to the significance codes.

Thus, in example summary GUI subsection 211 of GUI 205, the system may display standard medical terms linked to such significance codes based on such translation vectors and significance maps, discussed further below, in additional PRD indicators. And, in some embodiments, such PRD indicators may be presented in discrete subsection types, separated and/or organized spatially, to indicate the type of standardized information they present. In some embodiments, such discrete subsection types, separations and/or organizations are standardized across multiple PRDs, allowing healthcare providers to familiarize themselves and better access such information visually. For example, in some embodiments, a main symptom indicator 225 is provided, at or about the top of example summary GUI subsection 211 and, in similar PRDs for this or other patients, other indicators of main (or primary) symptoms reported for a different healthcare evaluation and/or patient, may be similarly placed at or about the top of such a GUI and/or GUI subsection. As mentioned above, in some embodiments, main symptom indicator 225, being provided within summary GUI subsection 211, includes standard medical terms linked to significance codes. In some embodiments, each such standard medical term is visually identified as being a standard medical term with an additional visual augmentation, such as example standard medical term indicator 227, example standard medical term indicator 229 and example standard medical term indicator 231, in the form of a surrounding box, font or (e.g., color) highlighting. In some embodiments, by “clicking on,” “tapping on” and/or otherwise selecting such a medical term indicator, the user (e.g., a healthcare provider authorized to access GUI 205) may access additional GUI tools presenting information related to the standard medical term within the indicator. For example, in some embodiments, the system may present an additional page of information and GUI tools related to the medical significance, triggers, comorbidities, conditions, diseases and other health information related to the standard medical term, as discussed elsewhere in this application. As another example, in some embodiments, the system may present a demonstrative tool, such as a pop-up arrow directed toward underlying verbal data, or an additional page of information and GUI tools related to the underlying verbal data, provided by the patient (discussed above) leading to the selection of the medical term indicated by the indicator.

In any event, in some embodiments, each of the standard medical terms so indicated within main symptom indicator 225 presents, in medical terms more familiar to the healthcare provider, the medical nature of the primary symptom being reported by the patient, in the context of the current provision of healthcare by the system and/or healthcare provider. In addition, if a standardized scoring related to that system has been provided as PRD by the patient (as discussed above), in some embodiments, an indicator thereof, such as example score indicator 233 may also be included within main symptom indicator 225. Thus, because the patient verbalized, in substance, the sensation of a sharp pain in his right shoulder each time upon performing the athletic act of serving while playing the sport of tennis, the medical terms within main symptom indicator reflect that information in standardized medical terms. And because the patient provided a PROMIS score of 8 out of 10 in severity with respect to this pain symptom, in response to a standard question from the healthcare provider, example score indicator 233 indicates that score as well (in some embodiments, with a differential score indicator, such as a bolded font, as pictured, or other unique visual augmentation or indicator.) In some embodiments, as one or more such PRD indicator(s) change over time (e.g., if the patient reports a new, different PROMIS score, for the same symptom), PRD indicator(s) are accompanied by one or more visual or other effect(s) (e.g., a symbol, filter or other outer or overall graphical augmentation), on, about or otherwise relating to the PRD indicator(s), which visual or other effects are based on such changing data. In some embodiments, such changes in PRD indicator(s) otherwise change in appearance, based on such changing data.

In some embodiments, qualifications of symptoms may also be displayed by additional GUI sub-tools, including visual representations of that relationship. Thus, as pictured, example main symptom indicator 225 is qualified by example onset and duration of symptom indicator 235, presented below, and to the right, of main symptom indicator 225, to which it relates, and includes a leading symbol (namely, a curved arrow, demonstrating a nested relationship) indicating that onset and duration of symptom indicator 235 qualifies main symptom indicator 225. Again, example onset and duration of symptom indicator 235 includes standardized medical terms and indicators thereof, similar to those discussed above, and with similar functionalities, in various embodiments.

In some embodiments, the system also presents standard medical terms identifying secondary symptoms, less centrally related to the reason for the patient's treatment or, in some embodiments, caused by or otherwise secondary to the main symptom, discussed above. Thus, for example, in some embodiments, summary GUI section 211 includes a secondary symptom indicator, such as example secondary symptom indicator 237. As with main symptom indicator 225, in some embodiments, secondary symptom indicator 237 may head additional, qualifying indicators, below, and to the right of it, such as example anatomical location indicator 239, which provides a medical term for the anatomical origin of the reported symptom (i.e., burning pain) reported by the patient. In some embodiments, a visual anatomical chart 241 may also be included, with lead line(s) 243 and/or other visual indicator(s) 245, of such an anatomical location(s), aiding the healthcare provider in rapidly acquiring location information related to the symptom.

It should be noted that, in some embodiments, a second, distinct form of the same reported symptom may be determined to exist by the system and/or healthcare provider. In some embodiments, the patient may be unaware of the distinct form of the symptom. For example, it is known that pain occurring with the same, or a similar, localization on a patient's body may be transmitted in different stages, due to different neural events. An acute injury (e.g., from an athletic act) may cause the perception of an immediate, sharp pain, mediated by pain receptors (nociceptors). Subsequent to that initial insult, a complex interplay of chemical signaling and inflammation may then occur, resulting in a longer, second stage of pain, reported as duller, burning pain, which may in fact relate to additional cellular and organ damage, due in part to that secondary inflammation process. But the patient ordinarily will only conceptualize a single injury, under such circumstances. In some embodiments, the system may identify such different symptoms from a report of a single symptom, and generate a secondary symptom significance code, and create an additional, secondary symptom indicator, based on that different symptom. Thus, in the example provided above, secondary symptom indicator 237 may be such a system-generated secondary symptom indicator, based on peer-reviewed, reproduced results of controlled medical studies, rather than relying on patients to differentiate and report such differentiated symptoms.

In some embodiments, narrative and historical information, not requiring standard medical terms, but providing color with respect to the etiology of symptoms and health conditions, may be provided in separate indicators, such as example narrative/historical etiology indicator 247. In some such embodiments, standardized language related to known activities may, instead of standard medical terms, be augmented with additional standard term indicators, such as example standard activity term indicators 249. In some embodiments, such standard activity term indicators may be of a unique form (e.g., highlight color, shape, animation), differentiating them from the standard medical term indicators, as discussed above.

In some embodiments, GUI 205 may include indicators of probable diagnoses for a patient—meaning, possibly applicable medical diagnoses based on the symptoms and other patient-reported data, and (in some embodiments) further analysis of the patient-reported data, as each are discussed above. In some embodiments, the system implements an algorithm expressing the probability of any of a number of potential patient diagnoses based on the prevalence of patient reported data in PRDs of other patients having been correctly given such diagnoses (e.g., as subsequently confirmed by re-diagnosis, or later confirming events).

Thus, in some embodiments, a probable diagnoses subsection 213 of GUI 205 is provided, including indicators of probable diagnoses 250 for the patient, based on the active PRD. For example, in some embodiments, such indicators include a most highly probable diagnosis indicator 251, indicating the potential diagnosis having the most likely application to the particular patient, based on the active PRD (explain active PRD as a component) and application of the algorithm. Other, less likely potential diagnoses may also be presented, for example, in additional probable diagnosis indicators 253. In some embodiments, an indicator of the level of probability of the respective probable diagnosis of the probable diagnosis indicator (e.g., such as example probability of diagnosis indicator 255), may be included within any or all of the probable diagnosis indicators.

In some embodiments, probable diagnoses and selections thereof are implemented through an algorithm created by supervised machine learning methods, for example, trained on data gathered, for the present patient subject to GUI 205, or other, similar patients, over a prior time period. In some such embodiments, such a machine learning algorithm is trained with the aid of a healthcare provider, who may label such prior observations of similar objects and activities as related to probable diagnoses of medical conditions in a prior time period. However, it is within the scope of the present application that such algorithms may be manually-created, by human software programming, or created by unsupervised machine learning methods.

In some embodiments, a healthcare provider may select one or more of the indicators of probable diagnoses 250 (e.g., by tapping on or otherwise selecting it, such as with any techniques for selecting GUI tools set forth in this application), based on her professional judgment and, in part, on a review of each and all of the indicators of probable diagnoses 250 listed within probable diagnoses subsection 213, recording it as her diagnosis of the patient's disease, disorder, or other health condition related to the symptoms in summary GUI subsection 211 and underlying material listed in freeform patient feed GUI subsection 215. In addition, in some embodiments, a healthcare provider, such as example healthcare provider 101, may implement treatments appropriate for such a diagnosis(es), for example, using any techniques set forth in this application, including any such digital therapeutics techniques, which may be subsequently prescribed by the healthcare provider to create or encourage behavior of the patient required by a treatment plan, or a discrete treatment, selected for the patient by the system and/or healthcare provider. In some embodiments, such subsequent digital therapeutics may include recommended interventions, reminders, instigations and any other tools aiding a user, such as patient 113, in carrying out such a treatment regimen requiring patient behavior. It should be noted that, although not specifically illustrated, in some embodiments, upon recording a diagnosis, as discussed immediately above, an additional GUI subsection of GUI 205 may be presented, including sub-tools indicating such prescribed treatment(s) and/or regimen(s) selected by the healthcare provider for the patient, and tracking a patient's progress as they undergo such a treatment and/or treatment regimen.

More generally, it should be noted that the number and types of subsections, and the number and types of listed GUI tools, and the number and types sub-tools (e.g., PRD indicators), and the amount and types of healthcare information presented and managed therein, as set forth above, are each illustrative, not exhaustive, of the nearly unlimited number, types and examples of potential subsections, GUI tools, sub-tools and healthcare information that may be included, in a wide variety of possible embodiments, each of which fall within the scope of the present application, and do not limit the scope of invention set forth herein, as will be apparent to those of ordinary skill in the art. For example, in some embodiments, GUI 205 includes a GUI subsection including GUI tools and sub-tools presenting and allowing the management of data and information gathered by the system (e.g., upon scanning or otherwise sensing relevant healthcare data related to the patient, such as vital signs). As another example, in some embodiments, GUI 205 includes a GUI subsection including GUI tools and sub-tools presenting and allowing the management of data and information input directly by the healthcare provider (e.g., in a narrative format, orally). The number and types of subsections, and the number and types of listed GUI tools, and the number and types sub-tools (e.g., PRD indicators), and type and amount of healthcare information set forth above are merely a reasonable set of examples set forth to aid in understanding the present invention. In a virtually unlimited number of alternative embodiments, a wide variety of alternative and/or additional GUI subsections, tools, sub-tools and healthcare-related information, fewer or greater in number, with fewer, greater, or different augmentations or data, in different orders, instances and having different capabilities, arrangements (e.g., underlying material placed on top, instead of the bottom), and other variations, with additional or alternative visual indicators of substantive relationships between tools and sub-tools, may be provided, other than the examples specifically set in the present application, and such additional and alternative embodiments also fall within the scope of the invention, as will be apparent to those of skill in the art. The examples set forth in the present application are merely examples, illustrating some principles of the invention.

It should also be understood, with respect to any figures and embodiments set forth in this application, that a wide variety of additional and/or alternative forms of tablet computer(s), smartphone(s), PDA(s), smartwatch(es), other peripheral device(s), control system(s), computer hardware, GUI(s), smartphone(s), and other device(s), GUIs, GUI tools, sub-tools, healthcare information, system(s) and method(s) and step(s) may be created, used or implemented, in different embodiments of the invention. Again, the exact number, disposition, arrangement, form and direction of GUI elements, tools, and peripheral devices provided herein are only examples of the myriad alternative and additional embodiments falling within the scope of the invention, as will be readily apparent to those of ordinary skill in the art to which the present invention relates. Similarly, as will be apparent to those of ordinary skill in the art to which the present invention relates, GUI 205, in general, may be formed in a wide variety of alternative shapes, sizes and dimensions, depending on the nature of the device on which it is presented, and may thus track a wide variety of additional, and different user, environmental, 3rd-party, research and other health-related data and information in various embodiments of the invention. For example, in some embodiments, such a GUI may include behavioral data (e.g., social interactions of the user), the user's heart rate, blood pressure, blood, skin or other bodily material analytes (e.g., via blood-testing hardware), and biomarkers, via similar or different GUI tools, as set forth above. In some such embodiments, the GUI and control system comprised within portable tablet computer 200 may instead be comprised within a form of bodily apparel (e.g., mixed reality glasses) or a wall-mounted or environmentally embedded computer, with other forms of display elements (e.g., via 3-dimensional (“3D”) display hardware) presented to user 100, instead of, or in addition to, smartphone 101. Again, the exact number, disposition, arrangement, form of peripheral device(s) provided herein are examples of the myriad alternative and additional embodiments falling within the scope of the invention, as will be readily apparent to those of ordinary skill in the art to which the present invention relates.

However, it should be noted that portable tablet computer 200 also includes a number of unique features, in some embodiments. For example, in some embodiments an actuable border, such as example actuable border 257, is included. As discussed above, in some embodiments, portable tablet computer 200 includes multiple, neighboring touchscreens 207, or, in some alternative embodiments, separately actuable touchscreen areas of PCD display 203. In some embodiments, when a user touches one of such touchscreens—namely, outermost touchscreen or touchscreen area 257, bordering an interior touchscreen or touchscreen area 259, but does not actuate such an interior touchscreen or touchscreen area 259, or another touchscreen or touchscreen area, in some embodiments (e.g., further out from the center of the PCD display 203), the system displays a previously undisplayed GUI or GUI aspect (such as a home screen, or a GUI dedicated to treatment options, in various embodiments) rather than the GUI presently pictured within interior touchscreen or touchscreen area 259. However, in such embodiments, a separate handle 261 may be included, away from outermost touchscreen or touchscreen area 257 and interior touchscreen or touchscreen area 259, to aid in such individual actuation of the two touchscreens or touchscreen areas. However, in some such embodiments, if a user touches another surface of PCD display 203, other than outermost touchscreen or touchscreen area 257, in conjunction with touching outermost touchscreen or touchscreen area 257, no such display of a previously undisplayed GUI or GUI aspect takes place. In some such embodiments, separate handle 261 may include a touch sensor or other sensor, and such an individual actuation feature is operable only if such a touch sensor or other sensor is so actuated by a user holding portable tablet computer 200 by the separate handle 261 and, in some embodiments, applying a minimums threshold pressure indicating substantially suspending portable tablet computer 200 by separate handle 261.

As mentioned above, in some embodiments, PRD indicators and other GUI tools and sub-tools may indicate changing information, over time, related to the healthcare of the patient. In some embodiments, such changes in indicators and other GUI tools and sub-tools may be accompanied by non-visual indicators and/or other effects. For example, in some embodiments, such GUI tools and sub-tools may be accompanied by audible sounds or sound effects, which audible sounds or sound effects may be altered based on such changing data. For example, in some embodiments, such audible sounds or sound effects accompany the user's viewing (e.g., determined via tracking the user's eyes as they point at one of the GUI tools) such a GUI tool or sub-tool. As another example, in some embodiments, such an auditory augmentation or effect is a sound effect emanating from, or simulating emanation from, the location of such a GUI tool or sub-tool.

In some embodiments, such GUI tools or sub-tools may be accompanied by tactile or haptic indicators and/or effects (i.e. “haptic feedback”), which haptic feedback may vary based on such changing data, in some embodiments. In some such embodiments, such haptic feedback may be a vibration and/or a pattern of vibrations. In some embodiments, such haptic feedback may be a tactile simulation of a surface. In some embodiments, such haptic feedback may be in the form of an electronic shock or other charge. In some embodiments, such haptic feedback may accompany the user's interaction with (e.g., touching) such a GUI tool or sub-tool. As another example, in some embodiments, such an auditory augmentation or effect is an effect emanating from, or simulating emanation from, the location of such a GUI tool or sub-tool.

In some embodiments, such GUI tools or sub-tools may be accompanied by olfactory or taste indicators and/or effects (i.e. “olfactory feedback”), which olfactory feedback may vary based on such changing data, in some embodiments. In some such embodiments, such olfactory feedback may be delivered by a scent disbursement actuator. In some such embodiments, such a sense disbursement actuator may combine and spray different amounts of source scent materials (e.g., terpenes), to deliver particular perceived scents associated with the GUI tools or sub-tools, or data or instructions thereof.

In general, any of the changes in appearance, sounds, indicators and effects, and/or additional effects related to a GUI tool or sub-tool may also relay representations of the changing health-related data that has been gathered and presented by the control system in GUI 205, in some embodiments. In some embodiments, such changes in appearance, sounds, indicators and effect, and/or additional, accompanying effects may relay aspects of that changing data.

In some such embodiments, the number, order, and combination of GUI tools and sub-tools selected by the control system may be based on an algorithm, as discussed further below. In some embodiments, such an algorithm incorporates at least some of such changed health-related data, as will be discussed in greater detail below.

Regardless of the form of the changed appearance, or other new or changed perceptible effects based on such changing data, such changes or new effects may be based on an algorithm related to the urgency of a patient's symptom, probable diagnosis or a treatment therefor, represented by the tracking indicator subject to such changes or new effects, in some embodiments. In some such embodiments, such an algorithm related to the urgency of the symptom, probable diagnosis or a treatment represented by the GUI tool and/or sub-tool may cause the control system to create such a changed location, appearance, or other new or changed perceptible effect based on the relative urgency of other GUI tools and sub-tools. In some embodiments, any of the above such changes or new effects are “changes in prominence” meaning that they alter the user's tendency to notice the tracking indicator or other indicator to which they relate.

In some embodiments, the changed prominence discussed above, or other changes in or relative to tracking indicators discussed herein, may be based on an algorithm other than an urgency algorithm. For example, in some embodiments, such an algorithm may be based on the control system's determination that certain health-related data is to be instigated, relative to carrying out an in-body experiment, as will discussed in greater detail elsewhere in this application.

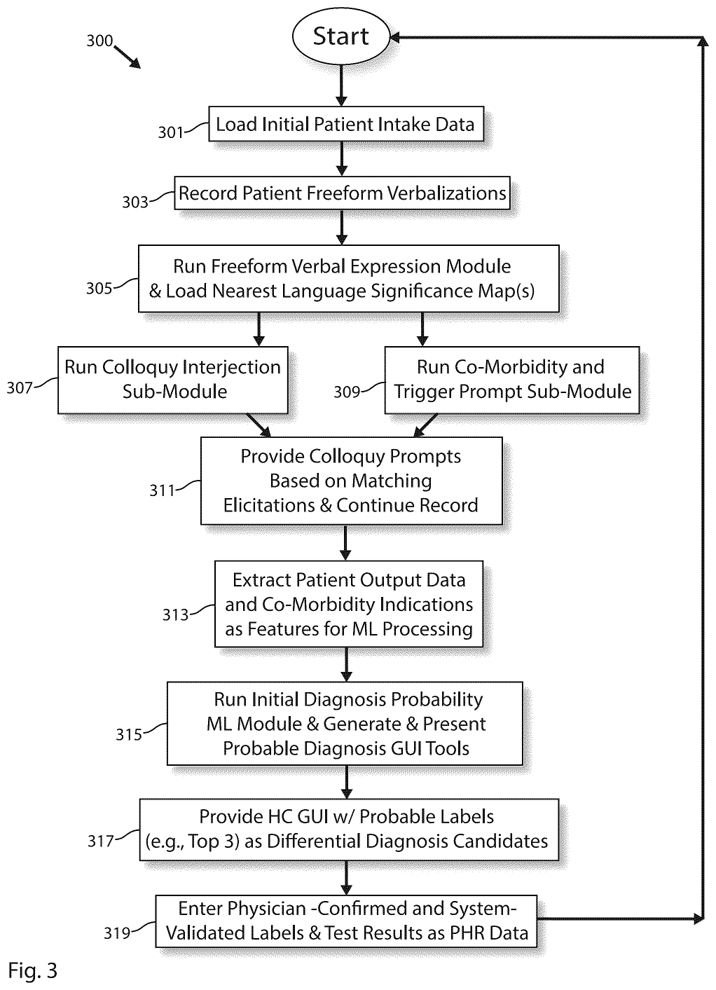

FIG. 3 is a process flow diagram, setting forth several example steps 300 that may be undertaken by a control system (such as the example control system set forth below, in reference to FIG. 5) implementing some aspects of the present invention, according to some embodiments. As discussed above, some devices, systems and methods set forth in the present application relate to GUI tools and other system-managed methods for recording PRD and other health-related data and information based on (e.g., verbal) input by patients, and recording standardized symptoms, diagnoses and treatments (e.g., Digital Therapeutics interventions) for a patient. The example steps set forth in reference to this figure illustrate some embodiments of how a control system, such as any of the example control systems set forth in the present application, including computer hardware and running computer software, might manage such data and operations. As will be readily apparent to those of skill in the art, a wide variety of alternative arrangements, steps, number of steps, sequences, and orders of steps also fall within the scope of the invention, and the exact steps, number of steps, sequences, orders of steps set forth herein are but one example, and do not limit the scope of the invention and disclosure.

Beginning with step 301, the system begins by loading data that may already be found within a patient's personal health record (“PHR”), for example, based on the patient's completion of a new patient intake form managed by the system (e.g., presented to the patient upon entry to a healthcare facility for a healthcare appointment). Typically, such a PHR may include past vital signs, demographic and identifying information, symptoms, diagnoses, ailments and other health conditions and concerns of the patient, among other information. In some embodiments, the system may load, access and use any and all such information within the PHR to aid in generating additional symptomatic information, probable diagnoses, treatments and other interventions, as discussed elsewhere in the present application, and in subsequent steps.

Proceeding to step 303, the system may next monitor the patient's verbal description, the patient's own words, of personal history, activities, ailments, symptoms and suspected or actual health conditions, and record such verbalizations as patient-reported data (“PRD”), as part of the patient's PHR, in some embodiments.

Next, in step 305, the system may run a sub-module of software and/or hardware specialized for analyzing ad hoc verbalizations from patients (i.e., a “freeform verbal expression module”). In some embodiments, as discussed above, from the patient's entire verbalization so recorded, the system may select and display certain terms and phrases of greatest relevance to the reason for the patient's visit to the healthcare facility, from the entire verbalization, based on an algorithm correlating patient verbalizations with particular symptoms related to such a reason, e.g., based on key terms and phrases identified as relevant to symptoms identified in recorded verbalizations and symptoms (e.g., validated symptoms, as set forth in this application) from past visits by similar patients to similar healthcare facilities for similar or related reasons. In some such embodiments, such a correlation algorithm may be created by machine learning techniques, as set forth in this application.

Similarly, in some embodiments, such a reason for the patient's visit may be recorded in the patient's PHR (e.g., intake form). In some embodiments, a healthcare provider may record such a reason for the patient's visit, based on her or his professional judgment. In some embodiments, the system may determine a most likely reason for the patient's visit for such recordation, based on correlations of key words and phrases (e.g., in a tag cloud) within the patient's verbalizations during the patient's present visit to the healthcare facility, e.g., based on applying a correlation algorithm, to other, similar patients' visits to similar healthcare facilities and verbalizations, and the most common reasons therefor. In some such embodiments, such a correlation algorithm may be created by machine learning techniques, as set forth in this application.

In some embodiments, the system may load significance maps, and apply translation vectors, as set forth in greater detail elsewhere in this application, to select and record standardized meanings derived from the patient verbalization. As also discussed elsewhere in this application, such significance maps may be generated and selected based on usage of human language by (e.g., demographically) similar patients, in similar ad hoc verbalizations while visiting similar healthcare facilities, for similar reasons. And such significance maps may be confirmed by independent testing for symptoms and health conditions in such other patients, as also discussed elsewhere in this application. Generally speaking, such significance maps may be generated by an algorithm, such as a machine-learning generated algorithm, correlating the usage of such particular key terms and phrases with such actually present symptoms and health conditions, in a large cohort of similar (e.g., demographically similar) prior patients.

In some embodiments, in subsequent step 307, the system may next run a sub-module of the freeform verbal expression module, also including specialized software, for assisting a healthcare provider in a colloquy with the patient, to aid in eliciting ad hoc verbalizations from patients (i.e., a “colloquy interjection sub-module”). In some embodiments, such a colloquy interjection sub-module provides a script (as discussed elsewhere in this application) including a sequence of questions, e.g., of a standardized form and order, to be presented to a patient (e.g., by a healthcare provider user of the system), in subsequent step 311, and record patient verbalizations in response thereto. As discussed elsewhere in this application, in some embodiments, such a script may be a dynamically-generated script, based on responses and other information provided to the system, and correlations between the ongoing verbalizations and probable diagnoses and related symptoms, in real time during the course of colloquy. In this manner, the system may more rapidly, reliably elicit relevant information. In some embodiments, such a script includes questions eliciting, or likely to elicit, standardized data (such as PROMIS scores). It should be noted, however, that it is also within the scope of the present application that the system may provide other forms of PRD elicitations, other than verbal questions (e.g., by presenting multiple-choice questions and answers, in a written format, or by observing symptoms via camera and/or medical sensor(s), as discussed elsewhere in this application).

In addition, in some embodiments, the system also proceeds, e.g., in parallel, to run a sub-module of the freeform verbal expression module, also including specialized software, to aid in identifying likely co-morbidities and symptom triggers for the patient (i.e., a “colloquy interjection sub-module”). In some embodiments, as with the colloquy interjection sub-module, the system may provide additional colloquy or other prompts in subsequent step 311 (e.g., via a GUI tool or sub-tool) for the patient or healthcare provider to inquire into the presence or absence of additional symptoms, not yet reported by the patient, but correlated or otherwise linked to a probable diagnosis, trigger and symptoms linked to or correlated with terms and phrases in the patient's ad hoc verbalization. In some embodiments, if the patient or healthcare provider then confirms the presence of such additional symptoms, the system may record such symptoms, and suggest additional probable diagnoses of such co-morbidities and triggers, as well.

In some embodiments, in step 313, the system may extract and implement the (e.g., anonymized) PRD so provided by the patient (e.g., in ad hoc verbalizations), and validated symptoms, triggers and final diagnoses by healthcare providers, as discussed below, to train a machine learning submodule, and similarly generate symptoms, triggers, comorbidities and probable diagnoses for similar (e.g., demographically similar) future patients.

Next, in step 315, the system may proceed to run a sub-module (e.g., a machine learning module) including specialized software generating an algorithm correlating potential diagnoses with the patient's ad hoc verbalizations and other PRD, and analyses based thereon (i.e., a “diagnosis probability machine learning module”). In some embodiments, a library of potential health conditions and diseases, and significance codes representative thereof, are stored and maintained by the system, and a plurality of (e.g., anonymized) other patients' PHRs including such diagnoses and PRD, if validated or finally made by an authorized healthcare provider, are used as a training set for such a machine learning module.

In some embodiments, the system may then present certain specialized GUI tools and sub-tools, incorporating standardized medical terminology linked to significance codes correlated with any terms and phrases within the patient's verbalizations, in a GUI presented to a healthcare provider, as set forth in the present application, in step 317.

Finally, in step 319, the system may validate one or more of the probable diagnoses, comorbidities and/or triggers, e.g., by objectively testing for the presence of such diagnoses, comorbidities and/or triggers, or by accepting the assignment thereof by a healthcare provider, as discussed elsewhere in this application.

Following any number of such sub-sets of steps, related to any number of aspects or effects for PRD recording and management GUIs, GUI tools or GUI sub-tools, the control system may return to the starting position.

Of course, in a virtually unlimited number of alternative embodiments, a wide variety of alternative and/or additional steps or processes, fewer steps or processes, different orders of steps or processes, instances of steps or processes, arrangements of steps or processes, and other variations of the steps and/or processes, with additional or alternative timing and preconditions, may be provided, other than the examples specifically set in the present application, and such additional and alternative steps and processes also fall within the scope of the invention, as will be apparent to those of skill in the art. The exact steps, number of steps, sequences, orders of steps set forth herein are but one example, and do not limit the scope of the invention and disclosure. The examples set forth in the present application are merely examples, illustrating some principles of the invention.

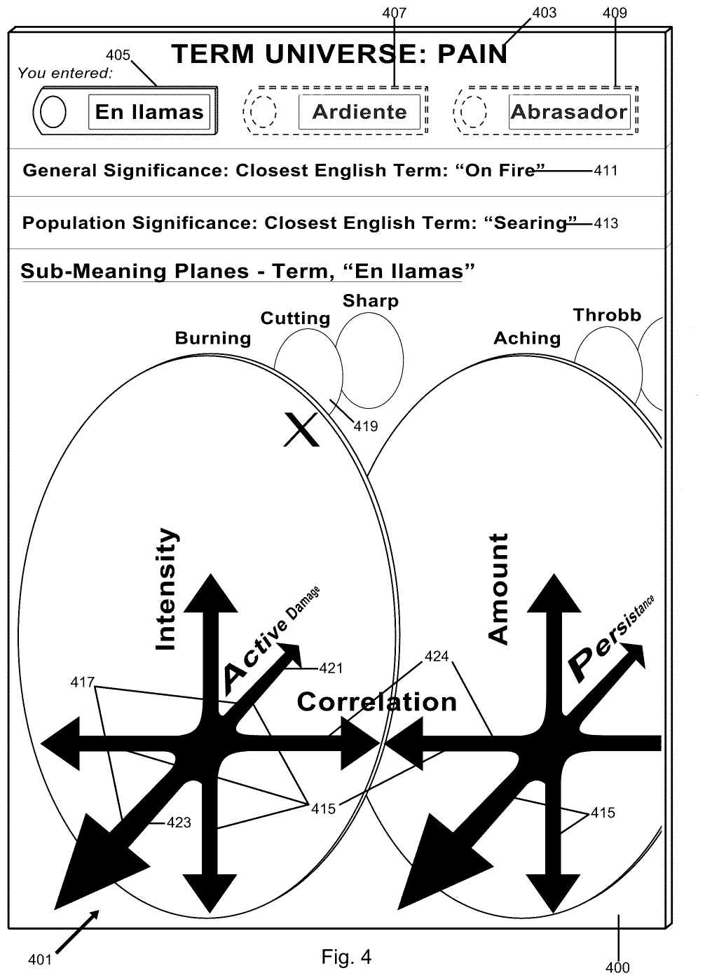

FIG. 4 is a front view of an example GUI 400 presented by a computer hardware and software control system, implementing some example aspects of the present invention related to monitoring and gathering data related to a user (e.g., patient-reported data/behavior), in accordance with some embodiments. As with other embodiments set forth in this application, GUI 400 may be presented and implemented through a display device and/or other computer hardware and software used in connection therewith (e.g., on a portable tablet computer smartphone or other PDA) in some embodiments. The example GUI 400 includes a depiction of example aspects of a Significance Map 401, which is a form of GUI tool configured for managing manual data entries and generating and recording standardized data by such a control system based on a wide variety of linguistic terms entered as input from a plurality of users. As used in this application, a “Significance Map” includes a plurality computer-based logical links between: 1) meanings and sub-meanings of a variety of human language terms and 2) language-neutral codes for new standard conceptual meanings related to a person (or other animal's) health. In some embodiments, as explained in greater detail below, when a user records information by inputting linguistic terms through the control system (e.g., in a GUI allowing for such data input as a basis for generating Digital Therapeutics) such a Significance Map represents the translation of that information into standardized data (a.k.a., a “Translation Vector”). In some such embodiments, such standardized data is then recorded by the control system, and then serves as a basis for algorithms and other software and hardware techniques for delivering Digital Therapeutics, as will be explained in greater detail below. In some embodiments, as discussed above, such significance codes are, in addition, linked to standard medical terminology, which may then be incorporated into GUIs, and GUI tools and sub-tools, as also discussed above.

The example Significance Map depicted in FIG. 4 relates to a general conceptual universe, as shown by universe code 403—namely, the conceptual universe of “Pain.” While in the English language, the word “pain” may be considered to mean something more broad, and with numerous differing, and potential specified senses set forth within dictionaries, the term “Pain,” as shown in universe code 403, is instead a code linked or otherwise associated by the control system with a variety of sub-codes, which, themselves, are associated by the control system to any conceptual meanings or sub-meanings relating to negatively perceived sensations or emotional feelings. Although the example universe code 403, bearing the code “Pain,” and some example sub-codes, conceptual meanings, and sub-meanings relating to negatively perceived sensations and feelings, are provided in and discussed with reference to the present figure, it should be understood that a wide variety of different codes and conceptual areas may, instead, be organized by a control system through any number of similar Significance Maps, related to any such universe codes, each of which Significance Maps and universe codes may be similarly managed by the control system, as set forth further herein.

In the example provided, a user may be entering data relating to the “Pain” code into the control system using GUI 400 using a term in his or her native language—in the example provided, the Spanish language. In some embodiments, the user may so enter data verbally, by speaking into a microphone—for example, upon a prompt by the control system to enter such terms in connection with creating a record of tracked sensations (among other health-related data recorded and tracked, and basing Digital Therapeutics treatments as set forth in this application). In some embodiments, a user may so enter such terms using a keyboard, mouse and/or touchscreen included within, or in communication with, the control system. In some embodiments, a user may enter such term(s) indirectly, and the term entry in created by the control system, based on other data related to the user's health and/or behavior (e.g., in some embodiments, if a user gasps through her or his teeth creating a hissing sound, after touching a flame or other high-temperature heat source, which are detected by microphones and cameras within the control system, and determined by the control system to be a behavior related to the significance of the term “searing”). In any event, regardless of the method of entry, the user has entered the Spanish term “en llamas,” as shown by example entered term indicator 405 within GUI 400, to describe a feeling which she or he is presently experiencing. A wide variety of other terms, and qualifying or localizing terms (locating the source of the pain referred to by the term on the user's body) may also, or alternatively, be used in such data entry by the user, in some embodiments, and the entry of this single term is, of course, merely one example.

Similarly, based on data collected from a wide variety of users of the control system (e.g., through different Software-as-a-Service accounts), many terms may be commonly used by those users to express similar perceived sensations. In some embodiments, the control system associates different terms, to different degrees (e.g., using a correlation algorithm) based on the number of instances of common usage. In some embodiments, such an association and/or algorithm is also based on users manually indicating (e.g., through a GUI aspect) that terms are associated. Terms so associated with such a term that is entered may provide sub-meanings of the term, in some embodiments. Thus, after a population of users has used a variety of pain-related terms over time, a Significance Map (in other words, an outline of meanings and sub-meanings of each term, and correlations and other relations thereof to other terms) is created by the control system—such as the Significance Map 401, which will be discussed in greater detail below. In some embodiments, the most closely related term(s) (e.g., with most strongly-correlated usage by the users) to each term may be recorded within and added to GUI 400. For example, in some embodiments, a series of closely related term indicators, such as term indicator 407 and term indicator 409, may be created and placed in GUI 400, presenting those closely related terms to the user—for example, on or about and/or abutting entered term indicator 405. As different terms entered by users are used more and less often by users of the control system, the exact terms presented in term indicator 407 and 409, and the number and rank of such term indicators, may change, becoming more accurate, and reflecting changes in usage by the population of users. In some embodiments, a user may “click on” or otherwise select either of term indicators 407 and 409, to enter the terms indicated within them (in this instance, the Spanish words “Ardiente,” indicated in term indicator 407, and/or “Abrasador,” indicated in term indicator 409), in addition to, or as an alternative to, selecting the term initially entered by the user (“En llamas”). In this way, the user may select terms that, upon reflection, and in consultation with the entire population of users, best expresses the sensation or emotional feeling she or he is experiencing, and record it with the aid of the control system.

In some such embodiments, a term most closely-related to the entered term may be determined by the control system and provided within GUI 400. In some such embodiments, a term in another language (other than Spanish, e.g., English) that is most closely related to the entered term may be so determined and provided—for example, as closest term indicator 411. As mentioned above, in some embodiments, such terms, the relation of terms may be based on correlated use between the entered term and its most closely related term within a population, as reflected by alternative closest term indicator 413. However, alternatively, or in addition, and as set forth in the instance provided as closest term indicator 411, such a closest term indicator may be based on accepted meanings as set forth by professional linguists (e.g., the authors of dual language or other dictionaries and/or other secondary sources of the significance of terms and words) and the correlation of term and word significance of different terms set forth therein. In some embodiments, an administrative user or other secondary user may presented with, and evaluate the significance of, the term entry by the user in one language, by being presented with a closest term indicator, such as the example provided as closest term indicator 411, or an alternative closest term indicator, such as the example provided as closest term indicator 413. In some embodiments, the control system may record both the initially entered term, and at least one of closest term indicators 411 and 413. In some embodiments, the control system may record both the initially entered term, and each of closest term indicators 411 and 413. In some embodiments, the control system may record the entry of such terms and associate a time of day, or other time period, with such an entry or pain sensation, in a database encoded with an account assigned to the user. In some embodiments, secondary users may review such recorded data and metadata, and such user accounts, if they are authorized to view data relating to the user.

In some embodiments, such different term indicators may indicate different meanings. For example, as pictured, closest term indicator 411 indicates that the closest English term to the entered Spanish term “En llamas” is “On Fire,” according to such secondary sources, but closest term indicator 413 indicates that the closest English term is actually “Searing,” according to correlation of use by the population of users.

In some embodiments, as with the universe code 403, any term entered by the user to signify an experience of Pain (by sensation or emotional feeling), as discussed in the example of “En llamas,” above, may be converted to a code, and a new, standardized significance related to that code. In other words, in some embodiments, rather than (or, in some embodiments, in addition to) recording the entry of the term “En llamas” merely as a term used in the Spanish language, the control system may enter “En llamas” as at least one code for a new, different, standard meaning managed by the control system. As mentioned above, such standardized meanings, and sub-meanings thereof, may be each so individually coded and correlated with one another, with their interrelations and degree of correlation recorded as a Significance Map, in some embodiments, such as example Significance Map 401.

For example, in some embodiments, a sub-meaning of the term “En llamas” is the concept that a burning sensation is sharp, and so sharp as to even be cutting, as experienced by the user. Because flames tend to concentrate their energy in narrow areas as fuel is burned, this relationship is literally experienced when a person is experiencing fire (e.g., accidentally licked by the tip of a fireplace flame) and, thus, such a localized, cutting sensation and association for the term “En llamas” may be commonly observed in a population. In some embodiments, such a sub-meaning may be assigned both to the code “En llamas” and to a sub-meaning, which may be coded as example sub-meaning codes “En llamas/Cutting” and/or “En llamas/Sharp.” In this way, if other users enter other terms, which also have such a standardized sub-meaning associated with it, the same code may be assigned to such data entry. As an alternative to such coding, or in addition to it, in some embodiments, a visual construct of such coding of such relationships may be presented to a user—for example, as a graph incorporating “lines” or “planes” of meaning, as illustrated by example lines of meaning 415. In some embodiments, such lines or planes of meaning are restricted to a single sub-meaning, which may be included within in any number of data entries by users (e.g., by different terms whose significance each include that sub-meaning.) In this way, a single term or code may be mapped, relative to others, which may share that sub-meaning. For example, as shown in example Significance Map 401, the term “en llamas” may share a sub-meaning, and illustrated line of meaning, that there is current, active damage to the user being perceived, which line of meaning is illustrated as example line of meaning 417. Similarly, a Significance Map for another term entered by users, namely “Cutting,” may be included within that line, but at a different location within the Significance Map, as shown by example neighboring Significance Map 419, shown in a minimized format, in the direction indicated (into the page, or “negative z” axis).

In some embodiments, a user may “navigate” between terms and codes sharing sub-meanings by “clicking on” one or more corresponding GUI arrow sub-tools, which may be provided in multiple directions along such a line of meaning. For example, line of meaning 417 is shown as including two such sub-tools—arrow sub-tool 421, for navigation in one direction, and arrow sub-tool 423, for navigation in a direction opposite to that one direction. In some embodiments, a line of sub-meaning may include a continuum of changing characteristic(s) of the sub-meaning. For example, as a user progresses in the direction of arrow sub-tool 421, the characteristic of a sharper active damage increases, such that, upon further navigation in that direction, the control system may present a more distant, albeit related, significant map 425, for the term “Sharp.” In some embodiments, a combination of one or more lines of sub-meaning significance may be referred to as a “plane” of sub-meaning, as illustrated by GUI planes 427, which may be comprised within a Significance Map. In some embodiments, Significance Maps are individually coded, recorded and modified over time, based on user data (such as the changing correlated sub-meanings of related, as discussed above).

In some embodiments, as with the lines of meaning, and GUI sub-tools dedicated thereto discussed above, Significance Maps may be closely related to one another within planes of meaning. For example, in some embodiments, users within the population of users managed by the control system may access, record or otherwise manage data encoded with a Significance Map in combination with access, record or otherwise manage data encoded with another significance map. In this sense, different Significance Maps, as with different terms, may be correlated with one another. In some embodiments, this correlation may be expressed as a line of meaning based on that correlation, such as correlation line of meaning 429.

In some embodiments, the user entering the term to record health-related data, or a secondary (e.g., administrative or authorized health professional) user may select or deselect such relationship, removing or recording their significance, and associating or disassociating them with the term entry by the user.

The totality of all Significance Maps managed by the control system, with all relationships between one another recorded, navigable, selectable and de-selectable, assisting in recording any known sensations or emotional feelings of the population of users, as set forth above, is referred to herein as a “Total Significance Map.”

FIG. 5 is a schematic block diagram of some example elements of an example control system 500, including computer hardware and preferably incorporating a non-transitory machine-readable medium, that may be used to implement various aspects of the present inventions, some of which aspects are described in reference to FIGS. 1-4 and 6 of this application, in accordance with some embodiments. The generic and other components and aspects described herein are not exhaustive of the many different control systems and variations, including a number of possible hardware aspects and machine-readable media, that might be used, in accordance with embodiments of the invention. Rather, the control system 500 is described herein to make clear how aspects may be implemented, in some embodiments.

Among other components, the control system 500 may include an input/output device 501, a memory device 503, longer-term, deep data storage media and/or other data storage device 505, and a processor or processors 507. The processor(s) 507 is (are) capable of receiving, interpreting, processing and manipulating signals and executing instructions for further processing and for output, pre-output and/or storage in and outside of the control system 500. The processor(s) 507 may be general or multipurpose, single- or multi-threaded, and may have a single core or several processor cores, including microprocessors. Among other things, the processor(s) 507 is (are) capable of processing signals and instructions for the input/output device 501, to cause a user interface to be provided or modified for use by a user on hardware, such as, but not limited to, a personal computer monitor or terminal monitor with a mouse and keyboard and presentation and input-facilitating software (as in a GUI), or other suitable GUI presentation system (e.g., on a smartphone touchscreen, and/or peripheral device screen, and/or with other ancillary sensors, cameras, devices, any of which may include user input hardware, as discussed elsewhere in this application with reference to various embodiments).

For example, in some embodiments, GUI tools, sub-tools, scanner(s), camera(s), microphones, or other sensor(s) and other user interface aspects may gather input from a user and present user(s) via colloquy (e.g., mediated by a health provider user) other verbal interactions (e.g., speech recognition and translation), observation techniques and/or with selectable options, such as preconfigured commands or data input and other GUI tools and sub-tools, to interact with hardware and software of the control system and monitor and manage a patient user's PRD and personal health, environment and data relevant thereto (e.g., food consumption, medication, other treatments and adherence to prescriptions and other such treatments, health-related personal regimens, and other user behaviors, biomarkers, data and extrapolations from those data, at particular times). For example, in some such embodiments, a user may interact with the control system through any of the actuation and user interface techniques set forth in this application, such as by verbal interaction and/or actuating tools and sub-tools of a GUI (such as any of the GUIs set forth in this application) to: record PRD (such as patient verbalizations related to symptoms), select and record significance codes as related to the patient's health and PRD, create and manage significance maps and translation vectors between patient verbalizations, significance codes, and standard medical terminology, identify probable diagnos(es) and select a diagnosis(es) therefrom, run experiments, record data related to the patient's personal health, behavior, consumption, biomarkers and environment, causing the control system to record those data and other extrapolations therefrom, or to carry out any other actions set forth in this application for a control system. The processor(s) 507 is/are capable of processing instructions stored in memory devices 505 and/or 503 (or ROM or RAM), and may communicate via system buses 575. Input/output device 501 is capable of input/output operations for the control system 500, and may include and communicate through innumerable possible input and/or output hardware, and innumerable instances thereof, such as a computer mouse(s), or other sensors, actuator(s), communications antenna, keyboard(s), smartphone(s) and/or PDA(s), networked or connected additional computer(s), camera(s) or microphone(s), mixing board(s), reel-to-reel tape recorder(s), external hard disk recorder(s), additional movie and/or sound editing system(s) or gear, medical scanners (such as CAT, PET or MRI scanning machines) or other medical sensors and/or computer hardware, speaker(s), external filter(s), amp(s), preamp(s), equalizer(s), filtering device(s), stylus(es), gesture recognition hardware, speech recognition hardware, computer display screen(s), touchscreen(s), sensors overlaid onto touchscreens, or other manually actuable member(s) and sensor(s) related thereto. Such a display device or unit and other input/output devices could implement a program or user interface created by machine-readable means, such as software, permitting the system and user to carry out the user settings and other input discussed in this application. Input/output device 501, memory device 503, longer-term, deep data storage media and/or other data storage device 505, and processor or processors 507 are connected with and able to send and receive communications, transmissions and instructions via system bus(es) 575. Deep data storage media and/or other data storage device 505 is capable of providing mass storage for the system, and may be a computer-readable medium, may be a connected mass storage device (e.g., flash drive or other drive connected to a U.S.B. port or Wi-Fi), may use back-end or cloud storage over a network (e.g., the Internet) as either a memory backup for an internal mass storage device or as a primary memory storage means, and/or may simply be an internal mass storage device, such as a computer hard drive or optical drive.

Generally speaking, the control system 500 may be implemented as a client/server arrangement, where features of the invention are performed on a remote server, networked to the client and made a client and server by software on both the client computer and server computer.

Control system 500 is capable of accepting input from any of those devices and/or systems set forth by examples 509 et seq., including, but not limited to—internet/servers 509, local machine 511, cameras, microphones and/or other sensors 513/514, Internet of thigs and/or ubiquitous computing device(s) 515, commercial or business computer system 517, and/or App-hosting PDA and related data storage device 519—and modifying stored data within them and within itself, based on any input or output sent through input/output device 501.

Input and output devices may deliver their input and receive output by any known means, including, but not limited to, any of the hardware and/or software examples shown as internet/servers 509, local machine 511, cameras, microphones and/or other sensors 513/514, Internet of things and/or ubiquitous computing device(s) 515, commercial or business computer system 517, and/or App-hosting PDA (such as, but not limited to a tablet computer, mixed reality headset, smartphone, or other suitable PDA known in the art) and related data storage device 519.

While the illustrated example control system 500 may be helpful to understand the implementation of aspects of the invention, any suitable form of computer system known in the art may be used—for example, a simpler computer system containing just a processor for executing instructions from a memory or transmission source—in various embodiments of the invention. The aspects or features set forth may be implemented with, and in any combination of, digital electronic circuitry, hardware, software, firmware, modules, languages, approaches or any other computing technology known in the art, any of which may be aided with external data from external hardware and software, optionally, by networked connection, such as by LAN, WAN or the many connections forming the Internet. The system can be embodied in a tangibly-stored computer program, as by a machine-readable medium and propagated signal, for execution by a programmable processor. Any or all of the method steps of the embodiments of the present invention may be performed by such a programmable processor, executing a program of instructions, operating on input and output, and generating output and stored data. A computer program includes instructions for a computer to carry out a particular activity to bring about a particular result, and may be written in any programming language, including compiled and uncompiled and interpreted languages and machine language, and can be deployed in any form, including a complete program, module, component, subroutine, or other suitable routine for a computer program.

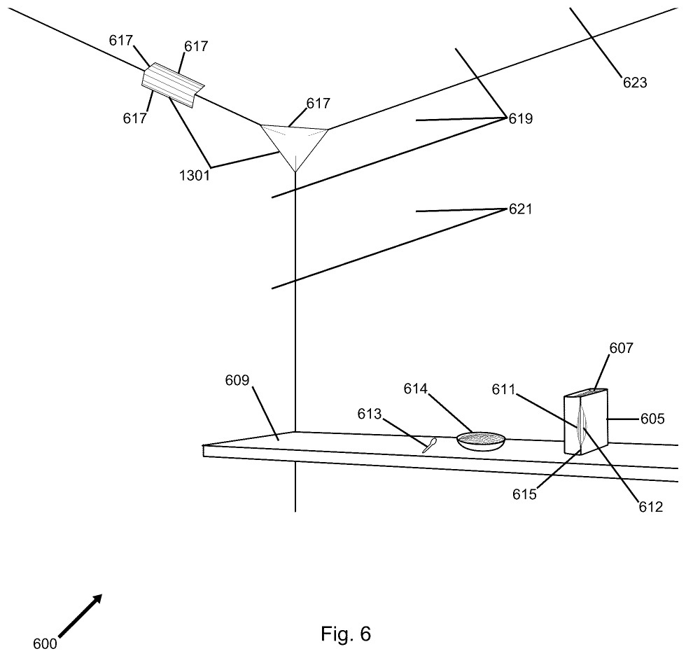

FIG. 6 is a perspective view of an example environment 600 in the process of being monitored by one or more example imaging sensor(s) 601, which may be controlled by a control system including computer hardware and software (such as any of the control systems set forth in this application), in accordance with some embodiments. In some embodiments, such an imaging sensor(s) 601 may be any suitable form of sensor for capturing an image and/or detecting and recording image data from an environment. For example, in some such embodiments, imaging sensor(s) 601 include a wide-angle imaging sensor, meaning that it is configured to take in a substantial proportion of the environment that it is placed in, on or about.

In some embodiments, such a wide-angle imaging sensor is capable of capturing an image of at least a 90-degree view of such an environment. In some embodiments, such a wide-angle imaging sensor is capable of capturing an image of at least a 120-degree view of such an environment. In still other embodiments, such a wide-angle imaging sensor is capable of capturing an image of at least a 180-degree view of such an environment. In still other embodiments, such a wide-angle imaging sensor is capable of capturing an image of at least a 270-degree view of such an environment. In still other embodiments, such a wide-angle imaging sensor is capable of capturing an image of at least a 360-degree view of such an environment.

In some embodiments, imaging sensor 601 includes at least one imaging, range-finding or other device for detecting the presence and/or nature of objects and/or activity within an environment. In some embodiments, imaging sensor 601 includes a camera. In some embodiments, imaging sensor 601 includes an infrared sensor. In some embodiments, imaging sensor 601 includes a rangefinder. In some embodiments, imaging sensor 601 includes a L.I.D.A.R. device. In some embodiments, imaging sensor 601 includes a R.A.D.A.R. device. In some embodiments, imaging sensor 601 includes a thermometer. In some embodiments, imaging sensor 601 includes a lens. In some embodiments, imaging sensor 601 and/or the control system managing imaging sensor 601 performs object recognition methods on image information it captures. As will be explained in greater detail below, in some such embodiments, such a control system maintains a library of data associated with particular objects or classes of objects, and compares image and other data it captures in real time with such data related to particular objects or classes of objects, thereby matching objects detected within an environment to particular objects or object types. As will also be discussed in greater detail below, in some embodiments, the control system analyzes image and other data captured by imaging sensor 601 in real time for the presence and changes in size of food items, medication, packages, contents, movement of a patient's body (to monitor exercise and/or the exhibition of symptoms) or other consumption and activity-related conditions, and then creates a record of such consumption and activity by a user. In some embodiments, the control system analyzes image and other data captured by imaging sensor 601 in real time for the presence and activity of a user (e.g., food consumption, exercise and symptoms), using similar comparisons to pre-recorded image and other data related to the user (e.g., facial recognition techniques). In some embodiments, the control system monitors a user's vital signs, biometrics, biomarkers or other indicators of the user's current health-related data, status or other condition. In some embodiments, the control system and/or imaging sensor 601 captures imaging data of substantially all physical activity of any matter viewable within an environment. In some such embodiments, imaging sensor 601 includes matter-penetrating imaging techniques (e.g., X-ray or ultrasonic imaging devices). In some embodiments, imaging sensor 601 includes a combination of two or more devices listed above.

In some such embodiments, and also as discussed in greater detail below, the control system may search and determine such matter, objects, conditions thereof and activities by users at a later time (e.g., by comparison to later-acquired object-, user- and activity-related data). In some embodiments, as discussed above, the control system may identify probable diagnoses of medical conditions (such as disorders and diseases) and potential causes, or complexes thereof, (a.k.a., hypotheses) from correlations of objects and activities detected in an earlier observed time, to conditions of a user, detected at a later-observed time. In some embodiments, such correlations, diagnoses and determinations are implemented through an algorithm created by supervised machine learning methods, for example, trained on data gathered, for the presently analyzed patient, or other, similar patients, over a prior time period. In some such embodiments, such a machine learning algorithm is trained with the aid of a healthcare provider, who may label such prior observations of similar objects and activities as related to probable diagnoses of medical conditions in a prior time period. However, it is within the scope of the present application that such algorithms may be manually-created, by human software programming, or created by unsupervised machine learning methods.

In some such embodiments, a repeated or otherwise strong correlation of such potential causes with such conditions of a user may give rise to higher priority probable diagnosis or hypothesis, which may be presented to a user and/or administrative user (e.g., a physician or other health care provider or other personnel).

As will also be discussed in greater detail below, in some embodiments, the control system manages a plurality of other such imaging sensors, similarly monitoring other environments, and objects, activities, patients, healthcare providers and other users therein. In some such embodiments, data related to environments, objects and users that are grouped together in some way may be linked and analyzed together in a single study (e.g., a retroactive experiment). In some embodiments, hypotheses developed, at least in part, from detecting one user's condition(s) and/or environment(s) may be presented to another user, based users' conditions and/or potential causes.

In any event, in the example pictured, environment 600 includes an example food container—namely, box 605 of granular food particles 607, placed on a kitchen counter 609. By observing box 1305 from a variety of angles over time, and passing related imaging data over time, with time stamps, to the control system, the control system can assess the amount of food present, the type of food present (e.g., by optical character recognition (“OCR”) of text on the box label hardware device 611, and/or by comparison to image data related to such food particles or types thereof stored in an object library) and the consumption of that food by a user (e.g., by user activity recognition). Such consumption and user activity recognition may be aided by control system recognition (e.g., via machine vision and/or additional artificial intelligence techniques) of ancillary objects (e.g., nearby consumption-indicating objects, such as example spoon 613 and example bowl 614). By observing the emptying of such consumption-indicating objects, in some embodiments, the control system may also determine a more precise time and rate of consumption of food particles 607 by a user (not pictured).

In some embodiments, box label hardware device 611 is a label comprising scannable hardware and information transmission technology. In some embodiments, such information transmission technology includes a code, such as a unique optical pattern 612, disposed on its outer surface. In some embodiments, box label hardware device 611 also includes a food scanning sub-device, disposed on an inside surface of the box label hardware device 611. In some such embodiments, such a food scanning sub-device is integral with, or disposed on, an interior surface of a food container, such as box 605. In some embodiments, unique optical patter 612 includes a control system including a dynamic display technology (e.g., an e-ink display) that changes to code for and/or reflect information regarding the contents of such a food container. In some such embodiments, such information regarding the contents includes a fill level of the food container. In any event, by scanning the unique optical pattern 612, sensors 601, and a control system comprising or comprised in them can readily determine the amount and type of food present within the food container, in some embodiments, at particular times. By assessing changes in such a fill level and/or contents, and the identity of a user present within environment 600 at those times, a food consumption rate, relative to the food present within the food container, can be determined. Base on such consumption rates, health-related data can then be recorded, and serve as the basis for Digital Therapeutics techniques and/or selecting significance codes (selecting a significance code related to the observed consumption, activity or symptom, and generating a PRD and/or GUI related to the same based on standard medical terminology based on such significance codes) set forth in this application. In some embodiments (as pictured) box label hardware device 611 is disposed on at least one corner or other vertex of a food container (such as the side box corner 615). In some such embodiments, the unique optical pattern is repeated on surfaces substantially disposed over multiple sides of box 605. In some embodiments, such a vertical pattern is not repeated on multiple sides of box 605, but is presented in a format visible from multiple sides of box 605. In any event, by presenting a unique optical pattern disposed from different sides of box 605, there is a greater likelihood that one or more of imaging sensors 601 will be able to sense, and obtain a reading of that unique optical pattern, which can then form the basis of Digital Therapeutics measures, as set forth in this application.

Of course, the example of a kitchen or other food consumption environment (environment 600) and food-related activity is just one of virtually unlimited possible environments and activities that may be similarly tracked in accordance with many alternate embodiments set forth in the present application. For example, in some embodiments, the environment observed may be a gym or other personal exercise environment, and the activity observed may relate to physical exercise, with observations of objects, materials and other indicators of such physical exercise. In other embodiments, the environment observed may relate to any particular human activity, objects or materials that is relevant to the health of a user.

Imaging sensors 601 may take on a wide variety of form factors, to enhance their operation, in addition to the form factors pictured. However, in some embodiments, multiple corner-filling formats are presented, some of which embodiments may include multiple (or all) distal ends or edges, such as the example edges 617, which taper seamlessly, creating a flush surface with, surfaces, such as example surfaces 619, of the walls 621, ceiling 623, or other surfaces of environment 601.

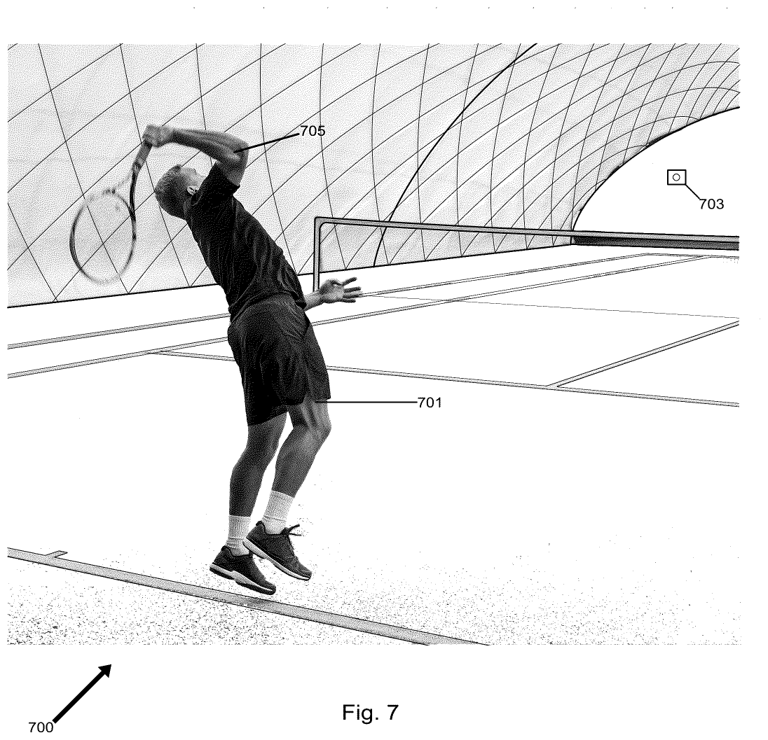

FIG. 7 is a perspective view of an example athletic environment 700, including a view of the same example patient 701, as discussed above, being monitored by an example imaging sensor 703 (which may be an imaging sensor similar in nature to that set forth above, in reference to FIG. 6), of personal health record generation system, similar in nature to any such systems set forth above, and including a control system, such as the example set forth above in reference to FIG. 5, in accordance with some embodiments.

In some embodiments, using example imaging sensor 703, and observational methods the same as, or similar to, those set forth above, e.g., in reference to FIG. 6, such a system may continue to monitor behavior, freeform verbalizations and other objects and events relevant to the health of patient 701, and to create patient-reported data based thereon, over time. Thus, in the example pictured, patient 701 is participating in the sport of tennis, attempting one or more serves of a tennis ball. In some embodiments, because the system recorded data related to the patient's experience of one or more symptoms (e.g., pain) upon playing the sport of tennis and, specifically, upon executing the movement of a serve (i.e., adduction), the system may create one or more records related to this activity and environment, as part of the data forming the patient's personal health records (PHRs). For example, in some embodiments, the system records the event of the patient exercising and, specifically, playing tennis and, more specifically, executing the one or more serves of a tennis ball. Even more specifically, in some embodiments, the system records the date and time of day that each of these occurred.

Furthermore, in some embodiments, the system may monitor specific movements of the patient's body while playing tennis, and determine and record additional information related to the patient's healthcare. For example, in some embodiments, the system maintains a library of model movements of the human body associated with playing tennis and, specifically, serving a tennis ball. In some such embodiments, by generally matching some of such movements to the act of serving a tennis ball, the system may determine that the patient is, in fact serving a tennis ball, and, more generally playing tennis. If, however, at least some of the patient's movements substantially deviate from such model movements for the act of serving deviations, the system may also record such deviations, and, in some such embodiments, may record a potential symptom and/or trigger (e.g., of inflammation) of the patient as part of the patient's PHR.

For example, in some embodiments, the patient may fail to complete a normal service motion, with the follow-through of the service motion omitted, for example. As another example, the patient may recoil, wince or flinch, creating an errant movement, deviating from the ordinary tennis service motion. In any event, in either of such examples, the patient 701's arm may fail to fully rotate, and remain locked in an unextended, recoiled position 705. In some embodiments, the system also maintains a library of such deviations as indicating potential pain triggered by athletic activity, based on the patient, and similar patients within a cohort (e.g., having tennis-related shoulder pain, and/or chronic pain and inflammation) having had similar recordings of similar deviant movements related to such conditions. And, in some embodiments, the system may record the patient's body movement(s) immediately prior to such a deviation as a potential trigger. In some embodiments, the system may record such pain and symptoms, and supplement such libraries, based on the patient themselves indicating that the deviating movement was related to pain, in some embodiments. In some embodiments, a healthcare worker, such as example healthcare worker 101, may be presented with such a recording of a video or symptoms, to review and evaluate the patient and the patient's reported PRD and PHRP, and make manual alterations to such records based on their professional judgment.

In any event, based on recording the pain and potential triggering activity, as additional experience with the patient, and data relevant to their healthcare, the system may create additional GUI tools for managing, diagnosing and treating the patient, as set forth in the following figure, in some embodiments.

FIG. 8 is a front view of the same example tablet computer 200, PCD display 203 and graphical user interface 205 of a personal health record generation system as set forth above, in reference to FIG. 2, but displaying additional user interface aspects, based on additional patient-reported data recorded by the system at a later time, based on additional experience with the patient, in accordance with some embodiments. As discussed above, in some embodiments, example portable tablet computer 200, may include a local control system (not pictured in the present figure) including PCD display 203 and an example graphical user interface of a personal health record generation system, in accordance with some embodiments. And, again, as with other control systems of PCDs, and other local control systems set forth in the present application, in various embodiments, local control system 201 may include, or be included within, a system for generating (e.g., eliciting, standardizing and recording) patient-reported data (e.g., personal health records) (the “system”), in accordance with aspects set forth in this application. As set forth elsewhere in this application, such a system may include specialized computer hardware and software of a control system that aids in eliciting, standardizing, and recording patient-reported data, creating personal health records, and managing medical interventions based thereon. Examples of such a control system, including such specialized computer hardware and software, are provided in reference to FIG. 5, below. Although the example of a portable tablet computer 200 is provided, any of the techniques set forth in this application may be practiced, instead or in addition, with other forms of PCDs and other such devices comprising, or comprised within such a control system, such as any of the other example types of devices set forth above.

As also mentioned above, in reference to FIG. 2, changes in location, appearance, sounds, indicators, other GUI aspects, and/or additional effects related to a GUI tool or sub-tool may also relay representations of the changing health-related data that has been gathered and presented by the control system in GUI 205, in some embodiments. In some embodiments, such changes in appearance, sounds, indicators, other GUI aspects an and/or additional, accompanying effects may relay aspects of that changing data.

And, also as discussed above, in some such embodiments, the number, order, and combination of GUI tools and sub-tools selected by the control system may be based on an algorithm, as discussed further below. In some embodiments, such an algorithm incorporates at least some of such changed health-related data, as will be discussed in greater detail below.

Regardless of the form of the changed location, appearance, order, or other new or changed perceptible effects based on such changing data, such changes or new effects may be based on an algorithm related to the urgency of a patient's symptom, triggers, comorbidities, probable diagnosis(es) and/or treatment(s) therefor, represented by the GUI aspect subject to such changes or new effects, in some embodiments. In some such embodiments, such an algorithm related to the urgency of the symptom(s), trigger(s), probable diagnosis(es) or a treatment(s) represented by the GUI tool and/or sub-tool may cause the control system to create such a changed location, appearance, or other new or changed perceptible effect based on the relative urgency of other GUI tools and sub-tools. In some embodiments, any of the above such changes or new effects are “changes in prominence” meaning that they alter the user's tendency to notice the tracking indicator or other indicator to which they relate.

In some embodiments, the changed prominence discussed above, or other changes in or relative to tracking indicators discussed herein, may be based on an algorithm other than an urgency algorithm. For example, in some embodiments, such an algorithm may be based on the control system's determination that certain health-related data is to be instigated, relative to carrying out an in-body experiment, as will discussed in greater detail elsewhere in this application.

In some embodiments, additional GUI tools and sub-tools are presented, based on such additional patient-reported data (PRD). For example, in some embodiments, as discussed above, such additional GUI tools and sub-tools include a new probable diagnosis indicator 807, new patient quotation sub-tools 809 and 811, new example standard medical term indicators, such as example standard medical term indicator 813 and other new patient-reported data indicators (PRDs). For example, patient quotation sub-tools 809 and 811 now omit mention of an acute injury, e.g., because the patient no longer associates the pain with a particular event. Based on usage of other patients, the terms used by the patient may now be matched more closely with significance codes, and medical terms, more closely associated with chronic, long-term pain, and a general, inflammatory condition, in some embodiments, and standard medical terms linked to such significance codes.

In addition to changing in appearance, type and substance, some of the PRD indicators and GUI tools within the figure, taken at a later date and reflecting such subsequent experience and recorded PRD, have changed in order and prominence, in accordance with the changing PRD. For example, the indicators of acute sharp pain, and burning, persistent pain, respectively, have reversed in order, with the persistent pain now being the primary symptom indicated, more toward the top of the GUI 205. In addition, the probability of diagnosis indicators have changed, and reshuffled in order, based on such changing PRD.

In addition, a new form of indicator—specifically, a trigger indicator 817, is now visible, within GUI 205, reflecting a potential ongoing relationship between a particular activity (serving a tennis ball, as discussed in FIG. 7) and similar increases in the patient's inflammation and at this later point in time, e.g., based on in-body experiments tracking a correlation between the activity, and subsequently reported shoulder pain (e.g., using the same or similar patient-reported terms and language usage). In some embodiments, such similarities are assessed with any of the algorithms correlating such language with validated symptoms and triggers, both for the patient, and other patients, of a demographic cohort including the patient.

It should be understood that the above-provided examples serve to illustrate certain healthcare and computer science aspects in accordance with some embodiments of the application, and are not exhaustive of the virtually unlimited set of like examples that fall within the scope of the present disclosure, as will be readily apparent to those of skill in the art in light of the disclosure. Each of the GUI, machine learning, algorithmic and methodological aspects set forth in this application may instead be carried out in a wide variety of alternative embodiments, in a wide variety of alternative and/or additional iterations and steps, different orders and arrangements of processes and elements, among other variations, with additional or alternative timing and preconditions, other than the examples specifically set in the present application, and such additional and alternative aspects also fall within the scope of the invention, as will be apparent to those of skill in the art. The exact examples set forth herein do not limit the scope of invention and disclosure. The examples set forth in the present application are merely examples, illustrating principles of the invention.

What is claimed is:

1. A system for generating personal health records, comprising:
a control system comprising computer hardware and software, comprising:
a freeform expression recordation and analysis module, configured to:
elicit, receive and record patient-reported data, including at least one first linguistic term(s) expressed by a first patient;
elicit, receive and record at least one second linguistic term(s) entered by other patient(s) and/or user(s), and generate a significance map, comprising at least one algorithm based on a common meaning between the usage of at least one of said first linguistic term(s) entered by said patient and at least one of said second linguistic term(s) entered by other patient(s) and/or user(s);
create and assign a unique significance code, comprising a standard clinical significance equivalent to a common meaning between said at least one first linguistic term(s) and said second linguistic term(s);
create a personal health record for said patient including at least one reference to said unique significance code.

2. The system for generating personal health records of claim 1, further comprising:
a diagnosis probability assessment module, including hardware and software configured to generate a list of potential diagnoses potentially relevant to the patient, and relative probabilities of each of the potential diagnoses having relevance to the patient;
a graphical user interface (“GUI”), comprising probability assessment sub-tools, each of which probability assessment sub-tools includes an expression of at least one of said relative probabilities.

3. The system for managing health-related data of claim 1, wherein said control system comprising computer hardware and software is configured to:
match each of said linguistic terms with an existing standardized meaning developed by the control system, and at least one existing unique code for said meaning assigned by the control system.

4. The system for managing health-related data of claim 2, wherein said control system is configured to:
store health-related data related to said user, related to said existing standardized meaning and related to said existing unique code.

5. The system for managing health-related data of claim 1, wherein said new standardized meaning and new unique code are assigned to more than one of said linguistic terms entered by said user and/or said other users.

6. The system for managing health-related data of claim 1, wherein said new standardized meaning and new unique code are assigned to a plurality of terms of different human languages and/or dialects.

7. The system for managing health-related data of claim 1, wherein said at least one algorithm comprises a plurality of relationships.

8. The system for managing health-related data of claim 6, wherein said plurality of relationships are incorporated into a Significance Map.

9. The system for managing health-related data of claim 1, wherein said new standardized meaning and said a new, unique code are based on a Significance Map.

10. The system for managing health-related data of claim 8, wherein said health-related data is stored via a Translation Vector.

11. A graphical user interface for recording and managing patient-reported data (PRD) and identifying probable diagnoses, triggers, comorbidities and/or treatments for the patient, comprising:
a freeform verbal expression sub-module, comprising specialized computer hardware and software configured to present a dynamic script to said patient and to record verbalizations from said patient;
a machine learning sub-module, comprising a neural network correlating PRD from other patients to said probable diagnoses, triggers, comorbidities and/or treatments.

12. A method for managing health-related data, comprising the following steps:
providing a control system comprising computer hardware and software configured to:
receive and record a first linguistic term entered by a user;
receive and record a plurality of other linguistic terms entered by the user and/or other users;
generate at least one algorithm based on relationships between the usage of at least one of said linguistic terms entered by a user and said other linguistic terms entered by the user and/or other users;
develop a new standardized meaning and assign a new, unique code for said meaning, and to associate said at least one of said linguistic terms with said code based, at least in part, on said algorithm;
store said health-related data related to said user, said new, unique code, and said meaning based on said new standardized meaning.

13. The method for managing health-related data of claim 11, comprising the following additional steps:
receiving and recording a first linguistic term entered by a user.

14. The method for managing health-related data of claim 12, comprising the following additional steps:
receiving and recording a plurality of other linguistic terms entered by the user and/or other users.

15. The method for managing health-related data of claim 13, comprising the following additional step:
generating at least one algorithm based on relationships between the usage of at least one of said linguistic terms entered by a user and said other linguistic terms entered by the user and/or other users.

16. The method for managing health-related data of claim 14, comprising the following additional steps:
developing a new standardized meaning and assigning a new, unique code for said meaning, and associating said at least one of said linguistic terms with said code based, at least in part, on said algorithm.

17. The method for managing health-related data of claim 14, comprising the following additional step:
storing said health-related data related to said user, said new, unique code, and said meaning based on said new standardized meaning.

18. The method for managing health-related data of claim 11, comprising the following additional step:
providing a Significance Map to a user, comprising a line of meaning and selectable sub-meanings related to said standardized meaning.
